(19) United States
(12) Patent Application Publication
(10) Pub. No.: US 2025/0025029 A1
(43) Pub. Date: Jan. 23, 2025

(54) NOVEL ILLUMINATING LARYNGOSCOPE AND METHOD OF INTUBATION

(71) Applicant: DJANOWOT PTY LTD, Highton (AU)

(72) Inventors: Edward L.B. WHITTEM, Highton (AU); Lucas Michael WHITTEM, Highton (AU)

(21) Appl. No.: 18/709,232

(22) PCT Filed: Nov. 11, 2022
(86) PCT No.: PCT/AU2022/051345

§ 371 Date: May 10, 2024

(30) Foreign Application Priority Data
Nov. 12, 2021 (AU) 2021903634

(51) Int. Cl. A61B 1/06; A61B 1/267; A61D 7/00; A61M 16/04; A61M 16/10

(52) U.S. Cl. CPC A61B 1/0615; A61B 1/267; A61D 7/00; A61M 16/0402; A61M 16/1005; A61B 2503/40; A61M 2250/00

(57) ABSTRACT

A laryngoscope and a method of using the laryngoscope in intubating mammals are described. The laryngoscope comprises a pen light grip for holding the laryngoscope like a pen, and an elongate probe attached coaxially via probe grip, wherein the elongate probe (14) is elongate and tapered such that the distal tip of the probe has the smallest cross section profile as compared to any cross section of the probe taken between the tip of the probe and the portion of the probe proximate the probe grip, and where the probe is curved and deflects away from the axis of probe and pen light grip and is substantially made from a material that transmits and or scatters light. The tip angle of deflection is 50 degrees and the deflection measured in mm is 50 mm. The tip 22 is twice as high as it is wide and, at the near tip point, the cross sectional profile is narrower than it is tall.

### CROSS-REFERENCE TO RELATED APPLICATIONS

The present application is a national phase application under 35 U.S.C. § 371 of International Application No. PCT/AU2022/051345 filed Nov. 11, 2022, entitled “NOVEL ILLUMINATING LARYNGOSCOPE AND METHOD OF INTUBATION,” which claims the benefit of and priority to Australian Patent Application No. 2021903634 filed Nov. 12, 2021, the contents of both of which being incorporated by reference in their entireties herein.

### TECHNICAL FIELD

The field of the disclosure lies in the field of medical instruments, including medical instruments used by veterinarians to intubate small animals. In particular, the disclosure relates to a laryngoscope that is designed to provide both a clear and direct view of the larynx and a patent pathway for endotracheal intubation.

### BACKGROUND

The following discussion of the present disclosure shall be largely described with respect to cats and, in particular small domestic cats. However, the disclosure is not so limited, and the skilled reader will appreciate that it would include other embodiments of the disclosure suited to other mammals, particularly other small animals of under 100 kg, including humans. A reference hereafter to a cat shall not be read in any limiting manner by the skilled reader.

In many species, after induction of general anesthesia it is necessary to establish an open airway, traditionally by passage of an endotracheal tube through the larynx and into the proximal trachea. This conventional procedure is depicted in FIGS. 1 to 4. To achieve this, it is usual to place the cat in ventral or lateral recumbency. An assistant grasps the maxilla and then raises the head by dorsiflexion of the neck. The clinician grasps the tongue and pulls it forward opening the mouth. This results in the cat having a presentation as depicted in FIG. 1. With one hand the clinician holds a laryngoscope device which is passed into the buccal cavity, the tip of which is placed firmly on the base of the tongue slightly anterior to the base of the epiglottis. The tip of the laryngoscope is pressed downwards and pulled rostrally, thereby opening the epiglottis and allowing visualization of the proximal larynx. Using the other hand, the clinician passes the lubricated endotracheal tube dorsal to the laryngoscope and into the proximal larynx, at which point visualization of the arytenoids and vocal cords is blocked by the tube. The endotracheal tube is then blindly pushed caudally into the trachea. FIG. 2 depicts a photograph of a cat being intubated by this conventional method. As can be seen the conventional laryngoscope and endotracheal tube approach does not provide good visualization to the veterinarian.

In additional this conventional approach to intubation of cats poses some complications. First, it is not always possible to adequately depress the epiglottis to enable full visualization of the larynx. Second, the soft palate can hang into the field of view obstructing visualization of the larynx. Most importantly, the endotracheal tube can stimulate touch sensors in the proximal larynx causing laryngospasm as FIG. 3 in which the spasmed larynx causes the air passageway to become closed whereby the cat will be unable to breathe. Finally, as the endotracheal tube is passed through the proximal larynx it can cause trauma to the mucous membranes, which cannot be visualized. The propensity to cause such trauma is increased in those species which are particularly prone to laryngospasm triggered by touch sensors in the proximal larynx, such as the domestic cat. Laryngospasm can cause difficulty passing the endotracheal tube. Laryngospasm also causes asphyxia, which if prolonged can cause morbidity and mortality.

Mitigation of morbidity and minimization of mortality due to asphyxia is attempted by providing pure oxygen to the patient for breathing for one to 5 minutes prior to induction of anesthesia. The patient or animal is then required to breath room air (approximately 20% oxygen) during the induction and intubation period. Better mitigation and minimization of morbidity and mortality could be achieved by providing pure oxygen directly to the pharynx and larynx during the intubation process, but currently used laryngoscope systems do not provide for this.

To overcome the problem of laryngospasm, it is common to apply to the proximal larynx a local anesthetic such as lidocaine. By paralyzing the superficial musculature of the larynx, it is hoped to prevent laryngospasm. This solution also has some problems. First, delivering a metered dose is important because of the narrow safety margin for these drugs, particularly in the domestic cat, and delivering a metered dose topically to a defined region of tissue is difficult. Second, after delivery of a metered dose there is a lag-time during which the drug is absorbed and begins to act. Current recommendations are to wait 1 to 2 minutes after drug delivery before attempting endotracheal intubation. Understandably, if the larynx is in spasm this waiting period causes one 1 to 2 minutes of asphyxia. The most common cause of anesthetic death in cats has been related to post anesthetic recovery laryngospasm and the resulting asphyxia. Post anesthetic laryngospasm is thought to occur as a result of traumatic endotracheal intubation.

Most laryngoscopy systems in current use also risk unintended spread of infectious disease between patients. Endotracheal intubation exposes equipment to the buccal cavity, pharynx, and bodily fluids. This equipment must be cleaned and sterilized as far as is possible between patients, but difficulty or error in cleaning exposes subsequent patients to risk for spread of infectious disease.

In some species, and in some individual animals with malformations of the buccal cavity such as dogs with Brachycephalic Airway Syndrome (BAS), the traditional laryngoscope and intubation method is blinded by the soft palate hanging into the field of view, obstructing clear visual and instrumental access to the opening of the larynx. In these animals it is necessary to elevate the soft palate dorsally to enable endotracheal intubation, and the traditional laryngoscope is not suited to this task.

In some species, such as humans and rabbits, the anatomy of the caudal pharynx makes passage of the endotracheal tube particularly difficult, because the tube must curve sharply ventrally to avoid accidental placement in the esophagus. Methods to overcome this difficulty traditionally include passage of a smaller wire stylet, and or passage of a nasoesophageal tube. These additional items help to deflect the endotracheal tube into the larynx. A simpler more reliable approach is needed which both deflects the endotracheal tube into the larynx, and allows visualization of that deflection, so as to give the practitioner confidence in the placement.

At the last step of endotracheal tube placement, the traditional laryngoscope and intubation method is blinded by the endotracheal tube at the last phase of placement as shown in FIGS. 2 and 6. To overcome this disadvantage, in some species, specialized stylets are passed into the trachea first to allow the endotracheal tube to be passed over the stylet, ensuring correct placement. This method fails to protect the mucous membranes of the larynx from trauma caused by forcible placement of the endotracheal tube. A method is desired that would allow full visualization of the open larynx during placement of the endotracheal tube.

Endotracheal intubation in humans is further complicated by the usual placement of humans in dorsal recumbency, causing the tongue to fall into the field of view. Therefore, laryngoscopes designed for humans need to deflect the tongue laterally to clear the view to the larynx. Because each patient differs from each other patient in anatomy, size, and shape, particularly between species, laryngoscope blades are usually exchangeable. Different designs of blade are available for different uses and/or to meet different preferences of practitioners, for example blades after Macintosh, Miller, Dorges and McCoy designs. A number of different length and different curvature of blades comprises a set, from which a suitable blade is chosen. Even where such different sets have been assembled for use in animals, most of these different blade designs originate from those designed for use in humans and few blade designs have been optimized for domestic animal species.

It is an object of the present disclosure to overcome or substantially ameliorate the problems of traumatic endotracheal intubation by providing an apparatus and a method for forming or using that apparatus.

### BRIEF SUMMARY

In the first aspect of the disclosure there is provided a laryngoscope for use in intubating mammals, the laryngoscope comprising:
a pen grip for holding the laryngoscope like a pen, and
an elongate probe, attached coaxially with the pen grip, wherein the elongate probe:
   is elongate and tapered such that the distal tip of the probe has the smallest cross section profile as compared to any cross section of the probe taken between the tip of the probe and the probe proximate the grip; portion of the
is curved and deflects away from the axis of probe and pen grip; s
is substantially made from a material that transmits light;
a light source that is directed to the probe and wherein light incident on the probe is transmitted or scattered in such a way that the probe becomes illuminated and emits light for aid in intubation.

Optionally, the pen grip incorporates the light source.

Optionally, the pen grip and the probe are able to be detached.

Optionally, the pen grip and probe are attached via frictional forces.

Optionally, the pen grip and light source is a penlight.

Optionally, the probe is hollow and made from translucent glass, acrylic, plastic or polymeric material.

Optionally, the probe is adapted to be heat sterilized or autoclaved.

Alternatively, the probe is adapted for single use and disposal.

Optionally, the probe has a grip portion that extends over the end of the pen light and attaches via frictional forces and a blade portion that extends from the grip portion, wherein the blade portion may include a straight portion extending between the grip portion and a curve start point of the probe.

Optionally, tip angle of deflection is between 5 degrees and 90 degrees and wherein the deflection measured in mm is between 5 mm and 90 mm.

Optionally, the tip angle of deflection is between 20 degrees and 70 degrees and wherein the deflection measured in mm is between 20 mm and 70 mm.

Optionally, the tip angle of deflection is 50 degrees and wherein the deflection measured in mm is 50 mm.

Optionally, the cross-sectional profiles of the curved portion of the blade of the probe are elliptical and either dorsally compressed or ventrally compressed or circular.

Optionally, the cross-sectional profile of the blade of the probe at its distal tip is compressed such that it is taller than it is wide to facilitate the probes insertion between the vocal cords or arytenoids of the mammal being intubated.

Optionally, the dimensions of the cross section of the distal tip are for the width, between 0.5 mm and 2 mm and for the height, between 1.6 mm and 4.0 mm.

Optionally, the dimensions of the cross section of the distal tip are 1 mm wide and 2 mm high.

Optionally, the cross-sectional profile of the blade flares out at the near tip position of the blade such that it is wider than it is tall which facilitates the opening of the vocal chords of the mammal being intubated.

Optionally, the dimensions of the cross section of the near tip position are for the width, between 2 mm and 8 mm and the height, between 1 mm and 4 mm.

Optionally, the dimensions of the cross section of the near tip position are 4 mm wide and 2 mm high.

Optionally, at the mid-point of the blade of the probe the cross sectional profile is flattened dorsally with a greater width than height.

Optionally, the dimensions of the cross section of the mid point position are for the width, between 4 mm and 10 mm and the height, between 4 mm and 10 mm.

Optionally, the dimensions of the cross section of the mid point position are 6 mm wide and 4 mm high.

Optionally, the near tip point is located between 70% and 99% of the deflected length of the blade and wherein the mid-point is between 10% and 60% of the deflected length of the blade.

Optionally the near tip point is located between 80% and 95% of the deflected length of the blade and wherein the mid-point is between 40% and 60% of the deflected length of the blade.

Optionally, the near tip point is located at 90% of the deflected length of the blade and wherein the mid-point is at 50% of the deflected length of the blade.

Optionally, the laryngoscope is hollow and includes: a connection for connecting a medical gas, a passageway through the probe to an aperture located near the distal tip of the probe which communicates the medical gas to the mammal during the use of the laryngoscope.

Optionally, the medical gas is oxygen and wherein the oxygen is delivered to the pharynx and/or larynx of the mammal.

According to a second aspect of the disclosure there is provided a method of using the laryngoscope of the present disclosure, comprising:
with a first hand, holding the grip of the laryngoscope in a pen grip;
passing the probe of the laryngoscope into the buccal cavity of the mammal and along the roof of the mouth in contact with the hard palate;
pushing the probe caudally, dorsal to the epiglottis, lifting the soft palate with the probe;
visualizing the proximal larynx;
applying local anesthetic to the larynx;
pushing the lubricated tip of the probe of the laryngoscope into the small fornix of the vocal folds between the arytenoid cartilages;
raising the tip of the probe dorsally whist pushing it caudally thereby opening the arytenoid cartilages;
using a second hand, passing a lubricated endotracheal tube ventral to (underneath) the laryngoscope and into the proximal larynx; and
withdrawing the laryngoscope as the endotracheal tube tip is passed gently between the arytenoids.

Optionally the method further comprises the preceding of:
inducing general anesthesia;
placing the mammal in ventral recumbency; and
grasping the tongue and pulling it forward presenting the open mouth for intubation.

Optionally, the method further includes administering oxygen or medical gas to the mammal that is performed throughout the intubation process via the opening in the probe connected to the source of oxygen or medical gas.

Optionally, the mammal is from the feline family.

Optionally, the mammal is a cat.

Alternatively, the mammal may be a rodent or canine.

### BRIEF DESCRIPTION OF THE DRAWINGS

Reference is now made to the figures of the specification in which the following is depicted:

FIG. 1 is a depiction of a cat's mouth ready for intubation.

FIG. 2 is a diagram showing the air passage open at the larynx of a cat.

FIG. 3 is a diagram showing the air passage closed at the larynx of a cat.

FIG. 4 depicts a cross section of a rabbit's head and throat showing the shape and geometry of the air passageways.

FIG. 5 depicts a cross section of a ferret's head and throat showing the shape and geometry of the air passageways.

FIG. 6 depicts a cross section of a dog's head exhibiting Brachycephalic Airway Syndrome (BAS).

FIG. 7 depicts a cross section of a normal dog's head and throat showing the shape and geometry of the air passageways.

FIG. 8 depicts a cross section of a human head and throat showing the shape and geometry of the air passageways.

FIG. 9 depicts a laryngoscope of the first aspect of the present disclosure.

FIG. 10 depicts a laryngoscope of the first aspect of the present disclosure being used in a first part of a method of intubation according to a second aspect of the disclosure.

FIG. 11 depicts the laryngoscope of the first aspect of the present disclosure being used in a second part of the method of intubation.

FIG. 12 depicts the laryngoscope of the first aspect of the present disclosure being used in a third part of the method of intubation;

FIG. 13 depicts a probe of the laryngoscope where various aspects of the probes shape and configuration are depicted including length, deflection, tip angle and mid point are depicted.

FIG. 13(a) is a cross section of the probe of FIG. 13 taken at the start of the curved portion of the probe (curve start point 13 of FIG. 13).

FIG. 13(b) is a cross section of the probe of FIG. 13 taken at the probe mid point (MP) (at point 25 of FIG. 13).

FIG. 13(c) is a cross section of the probe of FIG. 13 taken at the near tip point (point 28 of FIG. 13).

FIG. 13(d) is a cross section of the probe of FIG. 13 taken at the tip of the probe (point 21 of FIG. 13).

FIG. 14(a) to FIG. 14(g) are various views of probe 1515 including FIG. 14(a) side cross section, FIG. 14(b) rear view, FIG. 14(c) top cross sectional view, FIG. 14(d) perspective view, FIG. 14(e) close up view of the tip of the probe from FIG. 14(d), FIG. 14(f) cross section through line B-B of FIG. 14(a) and FIG. 14(g) is a cross section taken along line C-C of FIG. 14(a) where C-C represents the Tip Point 22.

FIG. 15(a) to FIG. 15(d) are various views of probe 1525 where C-C represents the Tip Point 22.

FIG. 16(a) to FIG. 16(d) are various views of probe 1535 where C-C represents the Tip Point 22.

FIG. 17(a) to FIG. 17(d) are various views of probe 1615 where C-C represents the Tip Point 22.

FIG. 18(a) to FIG. 18(d) are various views of probe 1625 where C-C represents the Tip Point 22.

FIG. 19(a) to FIG. 19(d) are various views of probe 1626 where C-C represents the Tip Point 22.

FIG. 20(a) to FIG. 20(d) are various views of probe 1628 where C-C represents the Tip Point 22.

FIG. 21(a) to FIG. 21(d) are various views of probe 1635 where C-C represents the Tip Point 22.

FIG. 22(a) to FIG. 22(d) are various views of probe 1715 where C-C represents the Tip Point 22.

FIG. 23(a) to FIG. 23(e) are various views of probe 1725 where C-C represents the Tip Point 22.

FIG. 24(a) to FIG. 24(d) are various views of probe 1735 where C-C represents the Tip Point 22.

FIG. 25(a) to FIG. 25(e) are various views of probe 2001 where C-C represents the Tip Point 22 and D-D represents mid point 30.

FIG. 26(a) to FIG. 26(e) are various views of probe 2002 where C-C represents the Tip Point 22 and D-D represents mid point 30.

FIG. 27(a) to FIG. 27(e) are various views of probe 2003 where C-C represents the Tip Point 22 and D-D represents mid point 30.

FIG. 28(a) to FIG. 28(e) are various views of probe 2004 where C-C represents the Tip Point 22 and D-D represents mid point 30.

FIG. 29(a) to FIG. 29(e) are various views of probe 2005 where C-C represents the Tip Point 22 and D-D represents mid point 30.

FIG. 30(a) to FIG. 30(e) are various views of probe 2006 where C-C represents the Tip Point 22 and D-D represents mid point 30.

FIG. 31(a) to FIG. 31(e) are various views of probe 2007 where C-C represents the Tip Point 22 and D-D represents mid point 30.

FIG. 32(a) to FIG. 32(e) are various views of probe 2008 where C-C represents the Tip Point 22 and D-D represents mid point 30.

FIG. 33(a) to FIG. 33(e) are various views of probe 2009 where C-C represents the Tip Point 22 and D-D represents mid point 30.

FIG. 34(a) to FIG. 34(e) are various views of probe 2010 where C-C represents the Tip Point 22 and D-D represents mid point 30.

FIG. 35(a) to FIG. 35(e) are various views of probe 2011 where C-C represents the Tip Point 22 and D-D represents mid point 30.

FIG. 36(a) to FIG. 36(e) are various views of probe 2012 where C-C represents the Tip Point 22 and D-D represents mid point 30.

FIG. 37(a) to FIG. 37(e) are various views of probe 2013 where C-C represents the Tip Point 22 and D-D represents mid point 30.

FIG. 38(a) to FIG. 38(e) are various views of probe 2014 where C-C represents the Tip Point 22 and D-D represents mid point 30.

FIG. 39(a) to FIG. 39(e) are various views of probe 2015 where C-C represents the Tip Point 22 and D-D represents mid point 30.

FIG. 40(a) to FIG. 40(e) are various views of probe 2016 where C-C represents the Tip Point 22 and D-D represents mid point 30.

FIG. 41(a) to FIG. 41(e) are various views of probe 3001 where C-C represents the Tip Point 22 and D-D represents mid point 30.

FIG. 42(a) to FIG. 42(e) are various views of probe 3002 where C-C represents the Tip Point 22 and D-D represents mid point 30.

FIG. 43(a) to FIG. 43(e) are various views of probe 3003 where C-C represents the Tip Point 22 and D-D represents mid point 30.

FIG. 44(a) to FIG. 44(e) are various views of probe 3004 where C-C represents the Tip Point 22 and D-D represents mid point 30.

FIG. 45(a) to FIG. 45(e) are various views of probe 3005 where C-C represents the Tip Point 22 and D-D represents mid point 30.

FIG. 46(a) to FIG. 46(e) are various views of probe 3006 where C-C represents the Tip Point 22 and D-D represents mid point 30.

FIG. 47(a) to FIG. 47(e) are various views of probe 3007 where C-C represents the Tip Point 22 and D-D represents mid point 30.

FIG. 48(a) to FIG. 48(e) are various views of probe 3008 where C-C represents the Tip Point 22 and D-D represents mid point 30.

FIG. 49(a) to FIG. 49(e) are various views of probe 3009 where C-C represents the Tip Point 22 and D-D represents mid point 30.

FIG. 50(a) to FIG. 50(e) are various views of probe 3010 where C-C represents the Tip Point 22 and D-D represents mid point 30.

FIG. 51(a) to FIG. 51(e) are various views of probe 3011 where C-C represents the Tip Point 22 and D-D represents mid point 30.

FIG. 52(a) to FIG. 52(e) are various views of probe 3012 where C-C represents the Tip Point 22 and D-D represents mid point 30.

FIG. 53(a) to FIG. 53(e) are various views of probe 3013 where C-C represents the Tip Point 22 and D-D represents mid point 30.

FIG. 54(a) to FIG. 54(f) are various views of probe 4001 where C-C represents the Tip Point 22, D-D represents mid point 30 and E-E represents the Near Tip Point 28.

FIG. 55(a) to FIG. 55(f) are various views of probe 4003 where C-C represents the Tip Point 22, D-D represents mid point 30 and E-E represents the Near Tip Point 28.

FIG. 56(a) to FIG. 56(f) are various views of probe 4002 where C-C represents the Tip Point 22, D-D represents mid point 30 and E-E represents the Near Tip Point 28.

### DETAILED DESCRIPTION

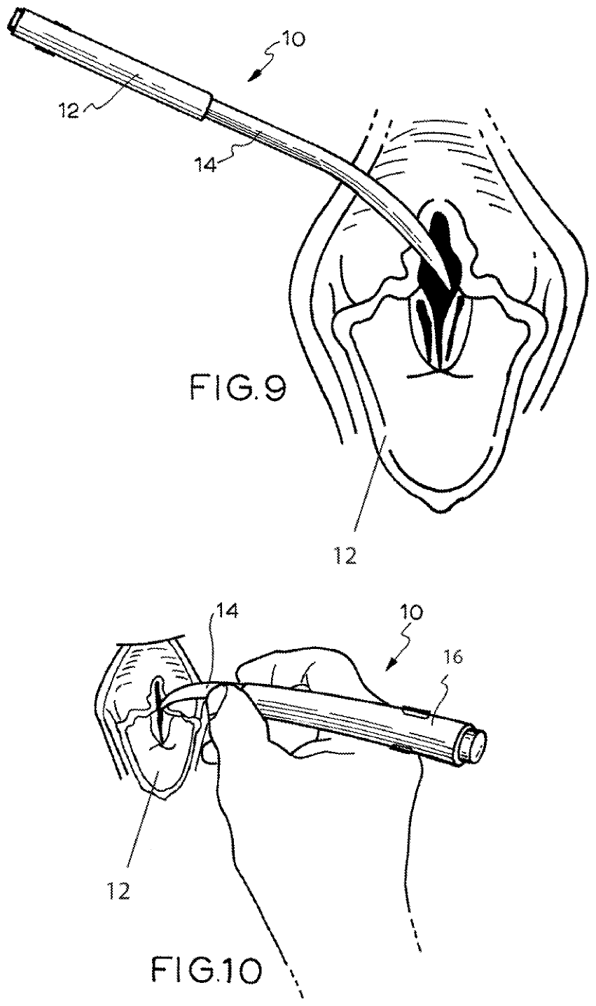

The first aspect of the disclosure is shown in FIG. 9 which depicts a laryngoscope 10 which is shown being used in a cat's mouth 12. The laryngoscope 10 is comprised of probe 14 connected to a light source and grip 12. The probe 14 is attached to a suitable light source and grip 16, such as a medical pen torch, by press fit using a suitable base. These include pen shaped lights (such as those marketed by LEDLENSER in West Ryde, Australia at www.ledlenser.com.au). Thin, pen shaped light sources 16 are recommended for the function of illuminating the probe 14 as the thin nature of the pen lights design facilitates them being held in a pen like grip which facilitates the carrying out of the method according to a second aspect of the disclosure.

The probe 14 can be made of any transparent or translucent material. Its manufacture is optimized to facilitate light transfer from the pen torch grip 16 internally through the probe 14, to illuminate the buccal cavity and larynx. The skilled reader will appreciate that only the distal end of the probe needs to be light transmitting. The grip end of the probe 14 does not need to illuminate as in the case of the other, distal end of the probe 14.

A probe 14 may be produced using many methods of manufacture, including injection molding or blow molding, and may be hollow or solid, and the material used for manufacture can be any suitable transparent or translucent material, and is optionally biodegradable, heat stable and chemically stable to enable cleansing and sterilization of a probe after use, if desired. Optionally, however, the material used is any transparent or translucent material which is biodegradable, and which allows the probe to be discarded after each single use, thereby not requiring cleansing or sterilization. The probe 14 may be made out of an inflexible material including acrylic or glass provided it is able to transmit light in the same way as the transparent or translucence polymeric material. Optionally glass or acrylic probes 14 would be frosted or made translucent to scatter light along its length. The probe may be provided with an internal structure including ribs and ridges or other reinforcing structures including cross members to provide additional mechanical strength.

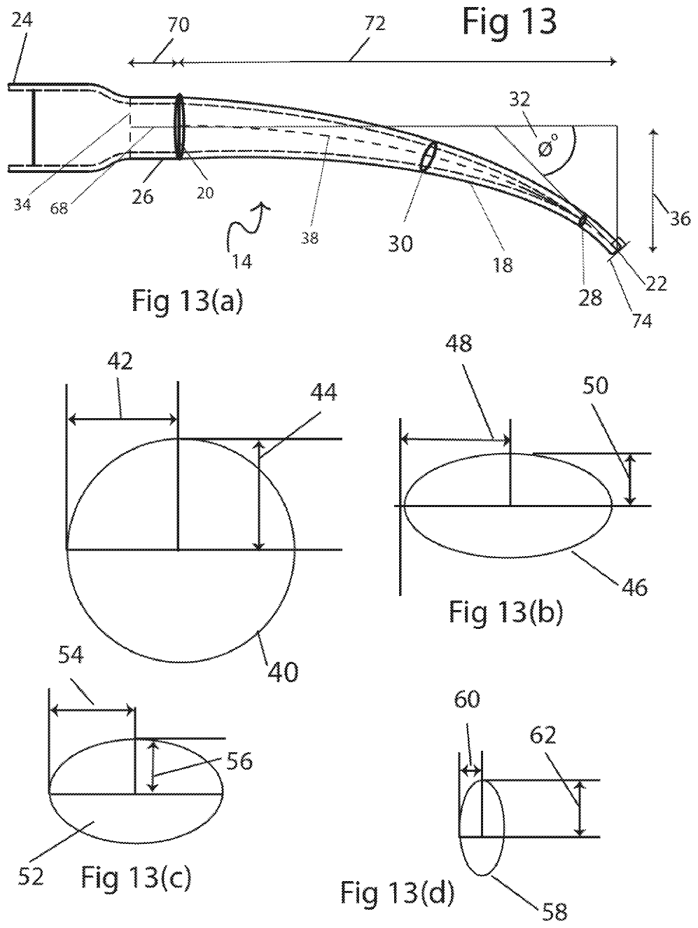

The main design parameters or elements of probe 14 are set out in FIG. 13 and FIGS. 13(a) to 13(f). The design specification allows for the production of the laryngoscope probes across a suitably wide range of parameters so as to be suitable for a variety of species and sizes of mammal. Surprisingly, it was found that a wide variety of shapes and sizes could be created with relatively few parameters to adjust including the length, curvature and cross-section profiles at the tip, near tip, the midpoint and where the blade 18 starts to curve.

Blade 18 is curved from the curve start point 20 to the tip 22 to optimize its passage along the hard palate, soft palate and into the larynx. Many shaped curves are suitable. We have found that a simple ellipse curve is suitable, but other curves such as Bezier curve or Basis-splines could also be provided in terms of the shape of the blade 18.

In FIG. 13 the probe 14 is comprised of:
a scope fit section 24 which is used to join the probe 14 to a light source and grip 6 and whose shape is dependent on what sort of light source is used; and
blade 18 wherein blade 18 is a long, tapered elongate projection that extends from the scope fit 24 and is curved; and
an optional straight portion 26 between blade start point 9 and curve start point 20 (between the scope fit 24 and the curved portion of the blade 18). This can be excluded if the size and geometry of the patient's mouth buccal cavity allows it.

The present inventors have discovered that a probe 14 according to the present disclosure can be simply defined by reference to the shape profile of the blade 18 at various specific positions along the length of the blade 18. Blade 18 has the following relevant parameters:
blade start point 34;
curve start point 20;
mid point 30;
near tip point 28;
tip point 22; and
tip angle Ø 32 which is measured as the angle of deflection from the center line or axis 68. Tip angle Ø 32 is measured at the point the center line 68 meets the straight line extending perpendicularly from tip tangent 74.

The skilled reader will appreciate that more points can be used to generate a more complex curve including with deflection points that fit the geometry of the patent and this shall also fall within the scope of the present disclosure.

Four other parameters have also been discovered to contribute to the working of the disclosure. These include (as depicted in FIG. 13):
deflected or curved length 38 is the length in mm of curved portion of blade 18 from the intersection of the curve start point 20 and mid line 68 to tip 22, that is, the actual curved length of blade 18, this length is used to define the mid point 24 and near tip point 23 along curved length 38.
undeflected length 72 in mm is measured from curve start point 20 to the tip 22 but along straight line 72 and not following the curve of the probe 14;
deflection 34 is the measure (in mm) of the degree of deflection between the tip 22 and the mid line 68; and
length of straight portion 26.

Each of these parameters can be adjusted to create a curved profile for the probe 14 which is a match for the subject patient's buccal cavity and geometry of the air passageways. Once the curve has been determined, the cross sectional shape of the various points on the blade 18 can be adjusted. FIG. 13(a) to FIG. 13(d) depicts these four cross sections at the following points of FIG. 13:
Tip 22;
Near Tip 28;
Mid Point 30; and
Curve Start 20.

Each profile has two aspects, half height and half width which is a measure of the cross section of each profile.

FIG. 13(a) is a cross section 40 of the probe 18 taken at point 20 of FIG. 13 in which:
measure 42 represents the Curve start point Half Width or CSPW; and—measure 44 represents Curve Start Point Half Height or CSPH.

FIG. 13(b) is a cross section 46 of the probe 18 taken at point 30 of FIG. 13 in which:
measure 48 represents the Mid Point Profile Half Width or MPPW; and—measure 50 represents the Mid Point Profile Half Height or MPPH.

FIG. 13(c) is a cross section 52 of the probe 18 taken at point 28 of FIG. 13 in which:
measure 54 represents the Near Tip Profile Half Width or NE;
measure 56 represents Near Tip Profile Half Height or NW;

FIG. 13(d) is a cross section 58 of the probe 18 taken at point 22 of FIG. 13 in which:
measure 60 represents the Tip Profile Half width or TPW;
measure 62 represents the Tip Profile Half Height or TPH;

The specific locations of curve start point 20, mid point 30, near tip point 28, tip point 21 of the embodiments disclosed in his specification are either depicted in the figure or are recorded in the tables of the specification where denoted by reference to % proportion. In the figures, a reference to B-B is a reference to the curve start point 20. The references to C-C in the figures denotes where the tip 22 is. The references to D-D in the figures is a reference to the mid point 30. The reference to E-E in the figures is a reference to the Near Tip point 28. The reference to percentage (%) of the curved length 38 is a reference to the position % along the curve of curved length 38. For example, a reference to 60% with respect to the curved length 38 means that this point is located 60% along the length of curved length 38 of blade 18 going towards tip 21.

In the embodiment of probe 14 in FIG. 13, Tip angle 32 is equal to 50 degrees. The undeflected length 72 is 70 mm and the deflection is 20 mm. There is also a uncurved portion 26 of 20 mm length. The mid point 30 is located 70% down the length of center line 38 of blade 18 starting from curved start point 20. The near tip point 28 is located 90% down along the deflected or curved length 17. At the Mid Point 30 the MPPH 50 is 2 mm and MPPW 48 is 3 mm. The NTW 54 is 2 mm and the NTH 33 is 1 mm. At the tip 21, the TPW 60 is 0.5 mm and the TPH 62 is 1 mm.

The profile of the probe 14 at the curve start point 20 was circular with a 5 mm radius. This was the case for all embodiments of the disclosure disclosed herein including those in subsequent examples. This was found to be adequate in terms of the ability remain rigid whilst hollow and transmit sufficient light. The lower bound for the cross section of the pre-curve start point 20 cannot be lower than approximately 2 mm radius for hollow embodiments, or sufficient light can't get through and/or the probe may break. In alternate embodiments the pre-curve profile at point 20 of the probe 14 could be made smaller by incorporating bundles of optical fibers to transmit sufficient light to the distal end of the probe where it is required and wherein the light source may comprise a thin flexible connection to a remote light source by further optical fiber.

The second aspect of the disclosure is the method of using laryngoscope 10 comprising a pen light 16 and probe 14. This aspect of the disclosure is best depicted in FIGS. 10 to 12.

The method for inserting an endotracheal tube benefits from good visualization of the larynx, using the device which also opens the arytenoids to facilitate easy passage of the endotracheal tube into the trachea. For best manipulative accuracy, the pen grip 16 is held by the clinician in the writing hand as though it were a pen, with the forefinger close to the point where the probe 18 meets the pen torch 16.

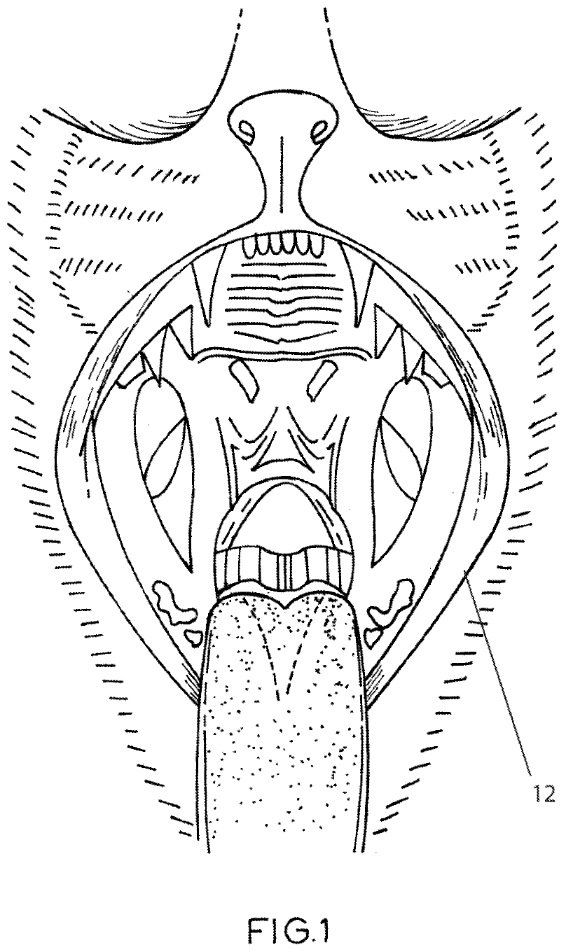

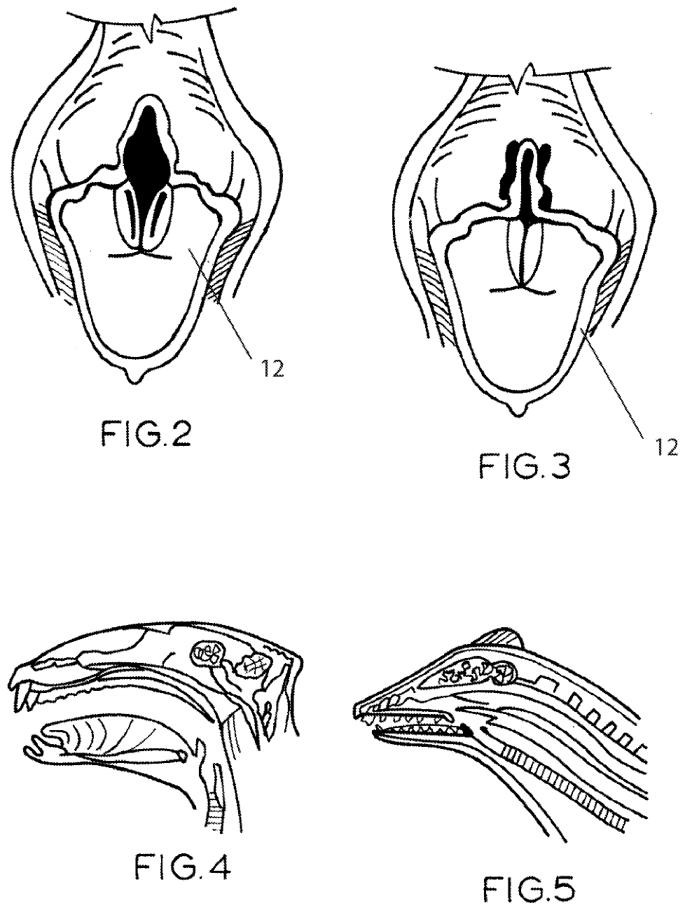

The method includes the general anesthesia being induced and the mammal being placed in ventral recumbency. Those skilled in the art will recognize that ventral recumbency is optional, but not essential. An assistant grasps the mammal's maxilla and then dorsiflexes the neck. The assistant then also grasps the tongue and pulls it forward presenting the open mouth to the clinician as depicted in FIG. 1.

FIG. 10 depicts the clinician passing the probe 18 into the buccal cavity, and along the roof of the mouth in contact with the hard palate, then continues to push the probe caudally, lifting the soft palate with the probe as shown in FIG. 11. The curved shape of the probe should approximate the curvature from the hard palate to the soft palate, and then dip toward the larynx.

A probe of dimension and curvature that matches the size and shape of the individual mammal is optional. The tip of the probe is passed dorsal to the epiglottis, lifting the soft palate. At this point visualization of the proximal larynx is achieved as shown in FIG. 11. Sometimes it is necessary to have the assistant gently extend the tongue a little further. At this point the larynx may be sprayed with any local anesthetic according to the preference of the clinician.

The end of the probe 18 is optionally shaped to improve its ease of passage through the small fornix or opening at the top of the vocal folds between the cuneiform processes. This is the space between the arytenoid cartilages. The lubricated probe tip is next pushed initially 1-2 mm through the opening at the top of the vocal folds in a gentle manner. The change in cross-sectional profile from the probe tip to the near end point (NE) 28 is to flare laterally: as this flared section of the probe tip gets to the opening, the clinician raises the tip of the probe dorsally while pushing it caudally, and this action opens and holds open the arytenoid cartilages, thereby opening the vocal cords.

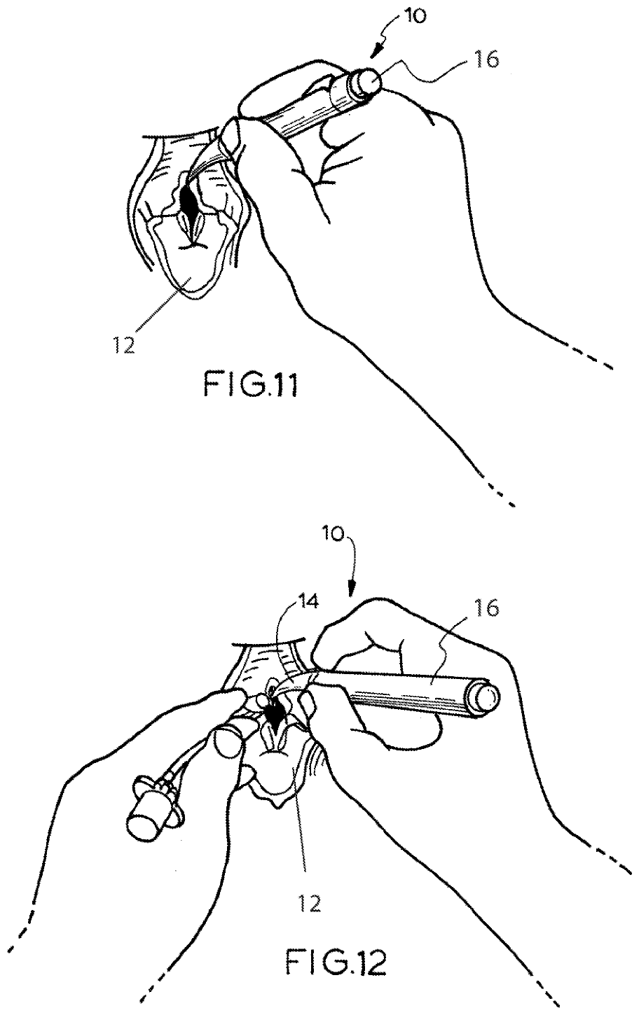

Using the other hand, the clinician passes the lubricated endotracheal tube ventral to (underneath) the laryngoscope and into the proximal larynx as shown in FIG. 12. As the endotracheal tube tip is passed gently between the arytenoids, the laryngoscope 10 is withdrawn.

It is important to note that whilst some of the following embodiments of the first aspect of the disclosure were desirable over others, all of the following embodiments of the first aspect of the disclosure worked and amounted to an improvement over the prior art apparatus and associated techniques.

### Example 1-Testing of Prototype Scopes 14 Made by 3d Printing Using Polylactic Acid as the Printing Substrate

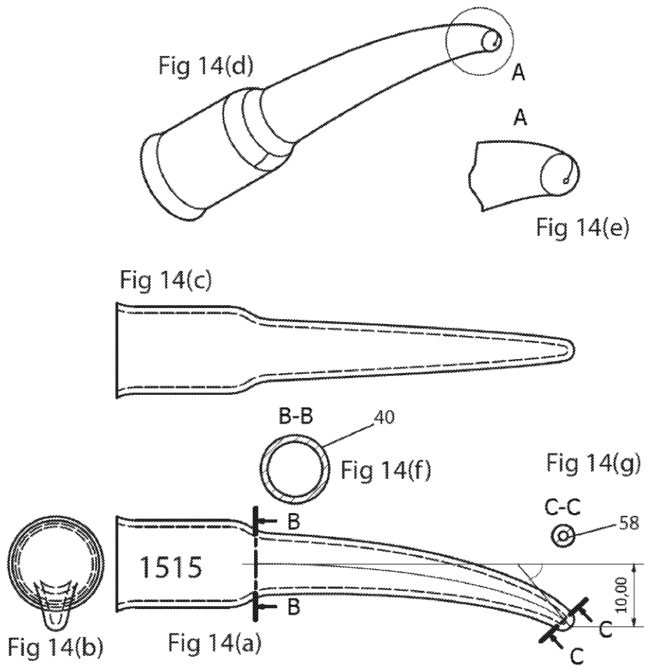

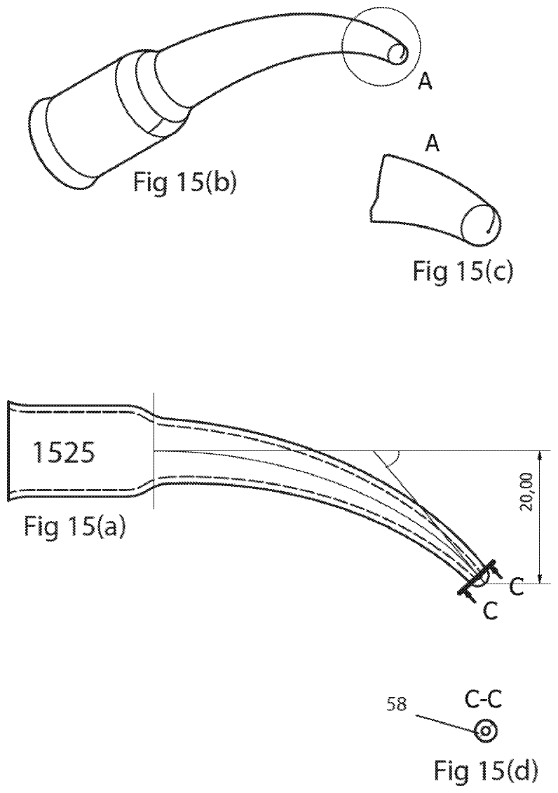

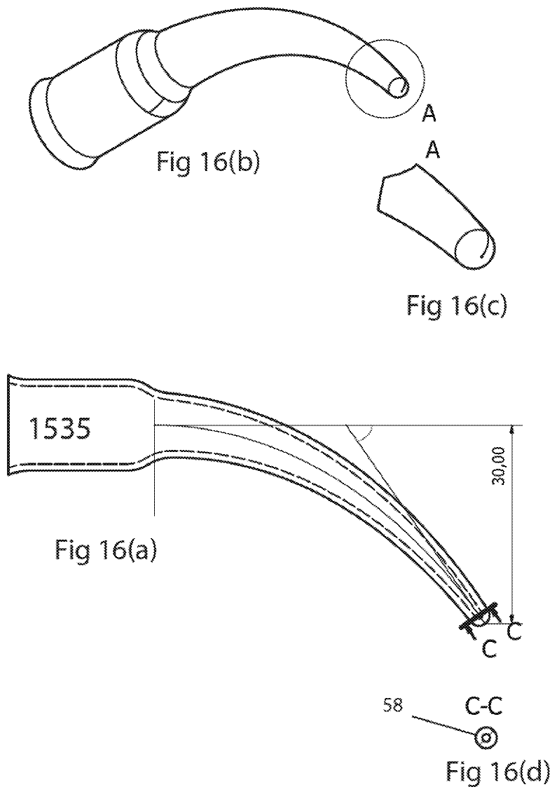

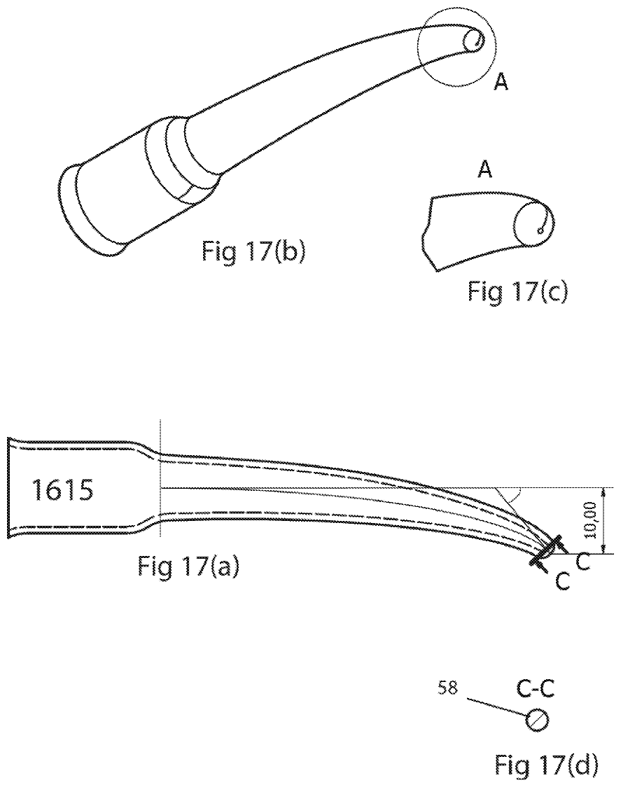

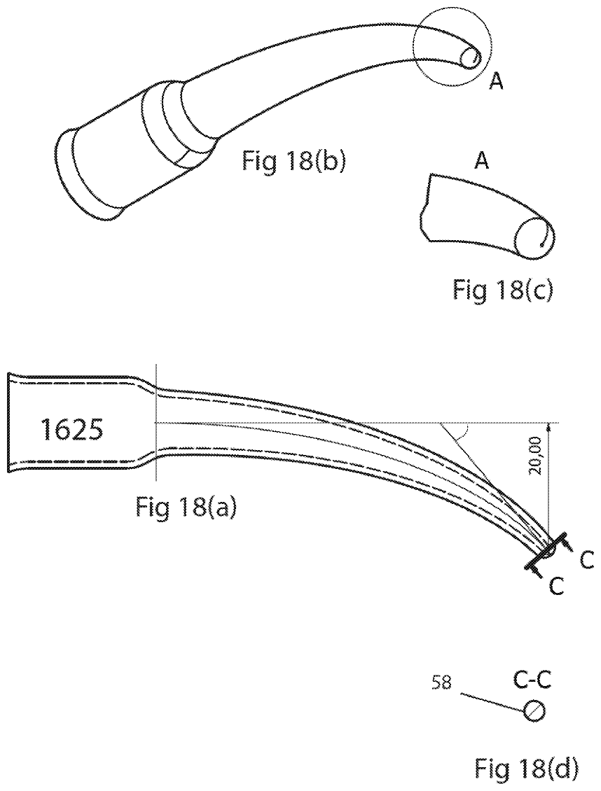

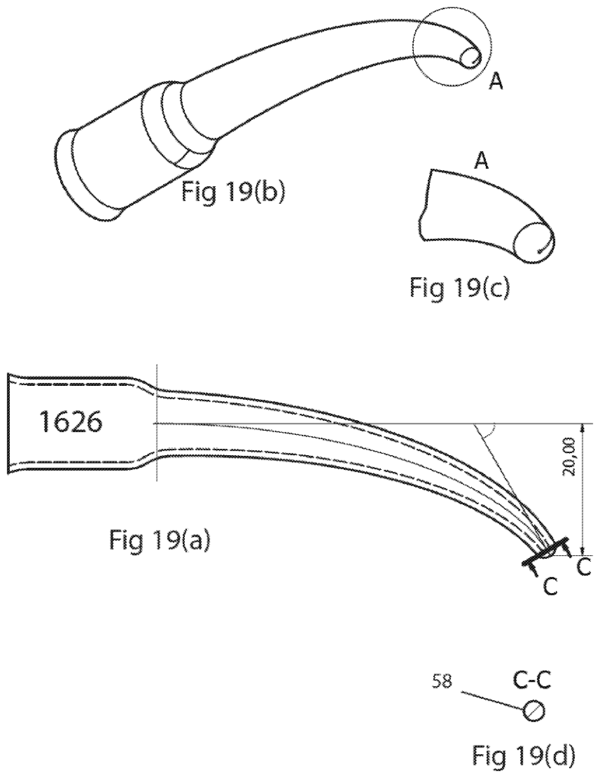

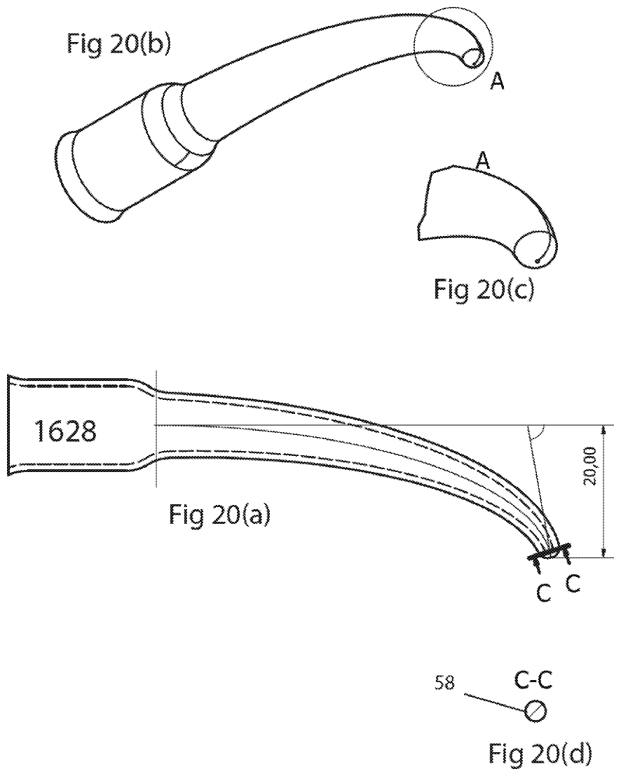

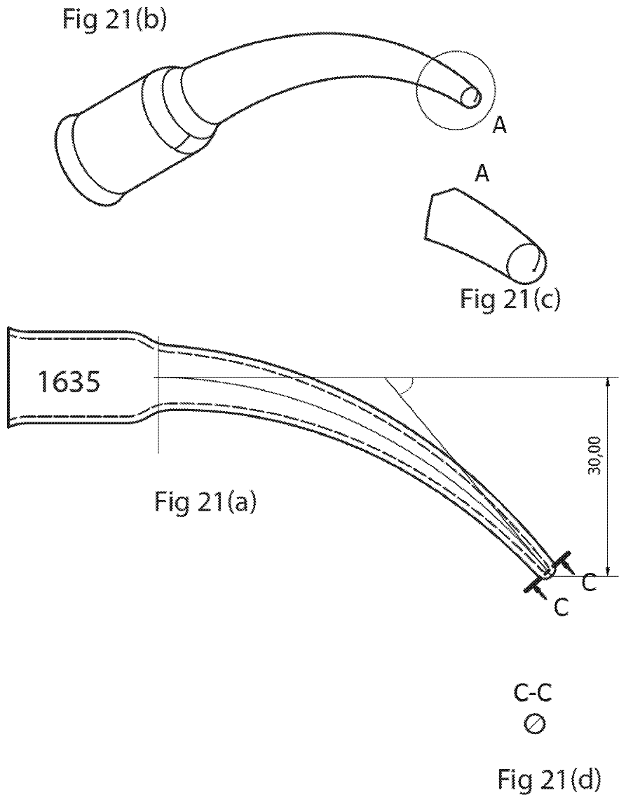

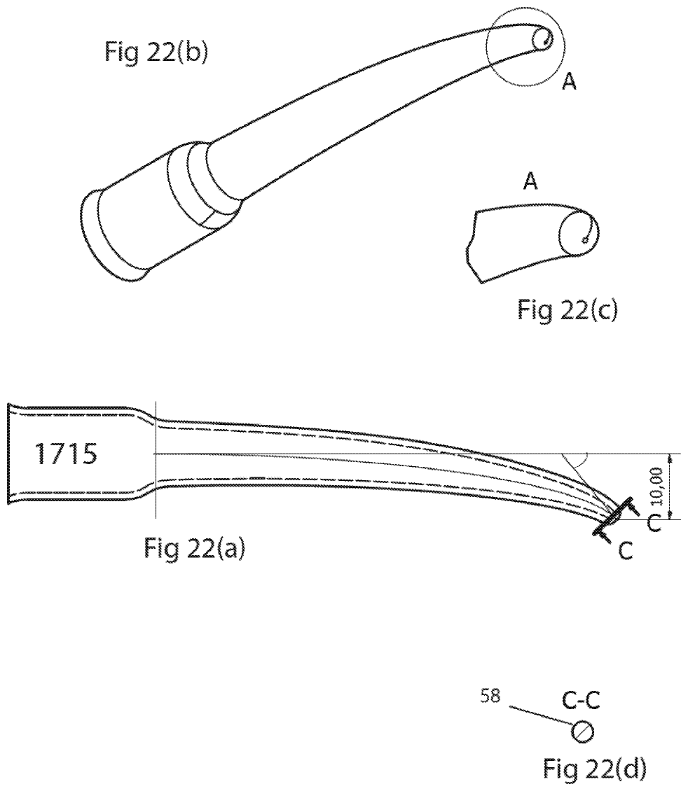

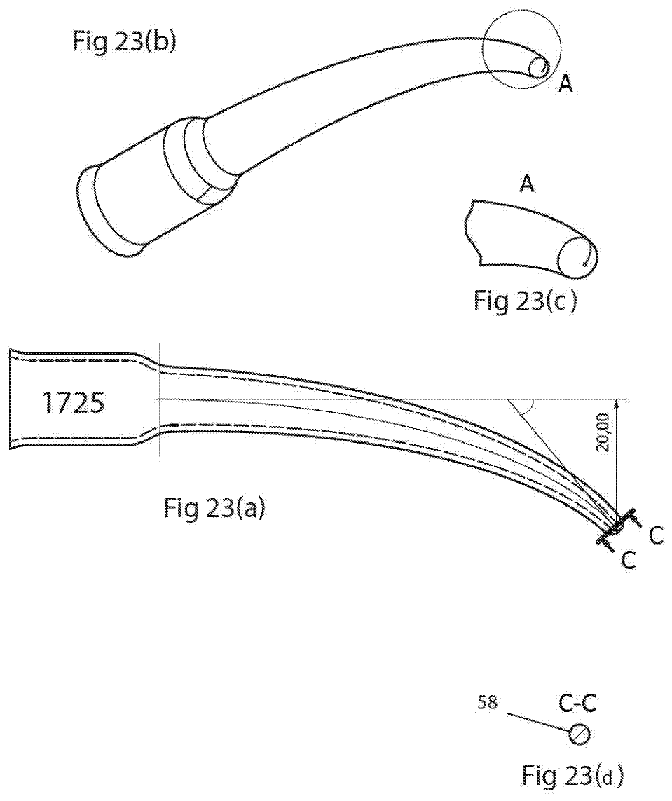

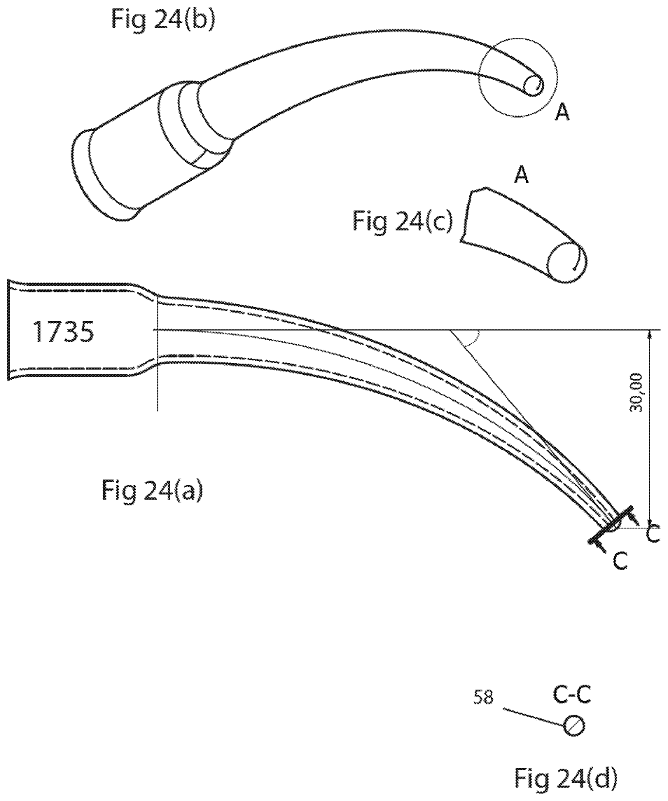

A total of 11 probes 18 were tested together with a light source inserted into the grip 24 of probe 18. Prototypes for the first example were prepared using a 3D printing substrate of transparent polylactic acid. The 11 probes printed are described in Table 2 and depicted in FIG. 14 to FIG. 24 respectively.

TABLE 1
parameters of probes tested

| | | | Tip | Mid-Point | | | | |
|---|---|---|---|---|---|---|---|---|
| | Length | Deflection | Angle | (30) | MPP-W | MPP-H | TP-W | TP-H |
| Prototype | (72) | (34) | (32) | % | (48) | (50) | (60) | (62) |
| ID | mm | mm | degrees | L(38) | mm | mm | mm | mm |
| 1515 | 50 | 10 | 50 | 50 | 3.5 | 3.5 | 1.5 | 1.5 |
| 1525 | 50 | 20 | 50 | 50 | 3.5 | 3.5 | 1.5 | 1.5 |
| 1535 | 50 | 30 | 50 | 50 | 3.5 | 3.5 | 1.5 | 1.5 |
| 1615 | 60 | 10 | 50 | 50 | 3.5 | 3.5 | 1.5 | 1.5 |
| 1625 | 60 | 20 | 50 | 50 | 3.5 | 3.5 | 1.5 | 1.5 |
| 1626 | 60 | 20 | 60 | 50 | 3.5 | 3.5 | 1.5 | 1.5 |
| 1628 | 60 | 20 | 80 | 50 | 3.5 | 3.5 | 1.5 | 1.5 |
| 1635 | 60 | 30 | 50 | 50 | 3.5 | 3.5 | 1.5 | 1.5 |
| 1715 | 70 | 10 | 50 | 50 | 3.5 | 3.5 | 1.5 | 1.5 |
| 1725 | 70 | 20 | 50 | 50 | 3.5 | 3.5 | 1.5 | 1.5 |
| 1735 | 70 | 30 | 50 | 50 | 3.5 | 3.5 | 1.5 | 1.5 |

Each probe was tested in a randomized sequence in a 13-year-old, 5.17 kg body weight, domestic shorthaired cat which was provided in a deceased state.

Each probe was rated for three outcome measures, each on three-point ordinal scale ranging from +1 to +3.

1. The first outcome measure was probe length, from too short (+1), good or correct (+2), and too long (+3).

2. The second outcome measure was a subjective evaluation of the adequacy of the curve shape or bend for the purpose of use, from not enough (or too straight) (+1), good or correct (+2), to too much (or too curvy) (+3).

3. The third outcome measure was a subjective evaluation of the location along the probe of the bend relative to the end of the probe (that is early or late bending) which is dependent upon the tip angle, from too rostral (tip angle too low) (+1), good or correct (+2), or too caudal (tip angle too high) (+3).

### Conclusions Drawn:

1. The length of the probe was important, and this was related to the size of the animal's head. This cat had a large head and therefore shorter probes may be more suitable in smaller animals such as rabbits or ferrets or juvenile cats.

2. Depending on the location of the bend, a longer laryngoscope probe could be quite suitable for smaller animals. Therefore, for domestic shorthaired cats, based on this one experiment, 60 or 70 mm length for probes may be favored.

3. How much curve occurs made a big difference to ease-of-use, with 20 to 30 mm of deflection being preferred.

4. Where the curve occurs was also very important for ease of access to the larynx ventral to the probe, i.e. where one must pass the endotracheal tube, as space is necessary. The location of the curve in each probe is largely determined by tip angle 32. The greater the tip angle 32 the “closer” the curve occurs to the tip 22.

5. The transparent PLA did not transmit light all the way to the tip as well as the translucent probes. It may be preferable to utilize translucent material that scatters the light from the light source 16.

### Example 2—Further Prototypes Tested

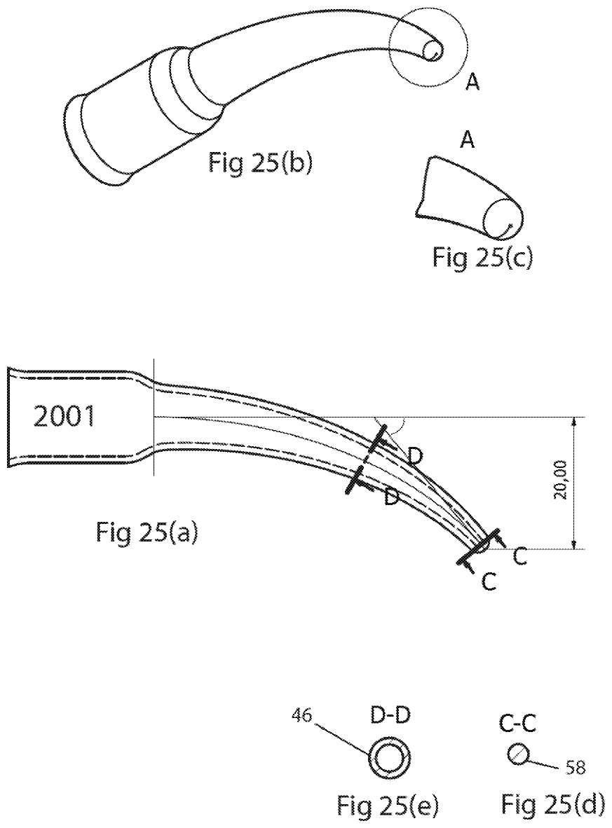

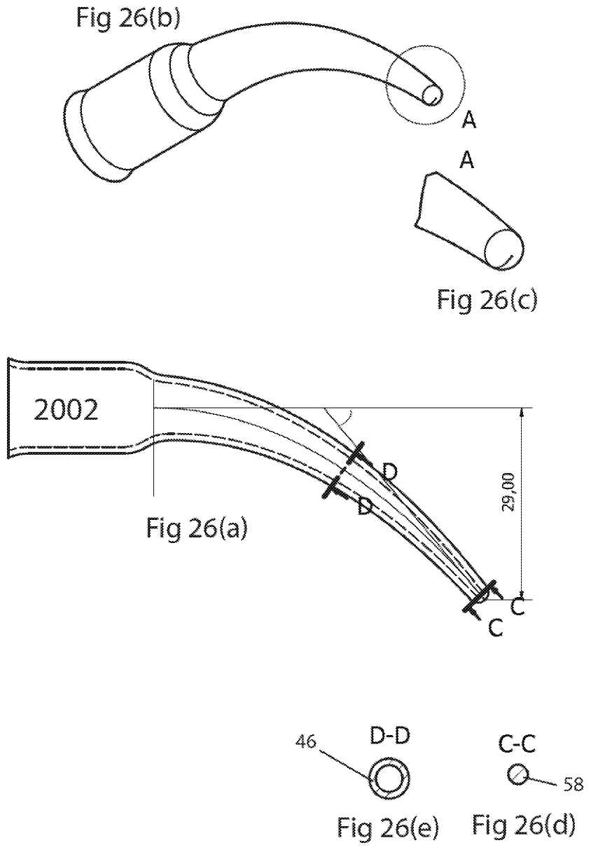

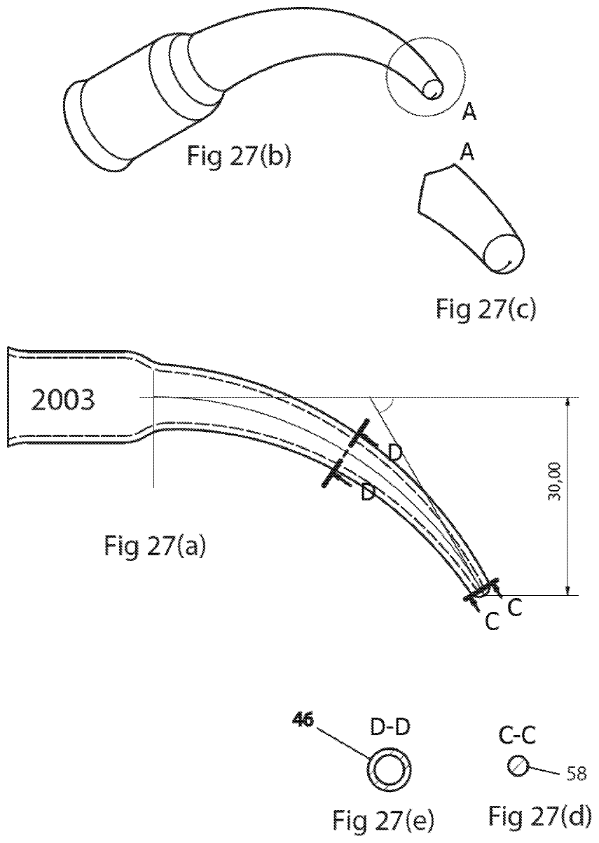

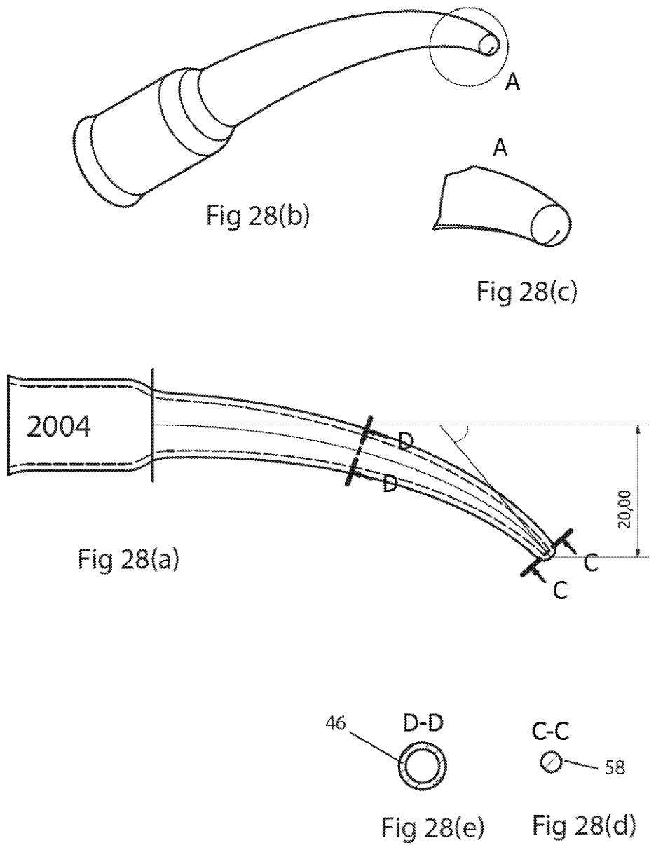

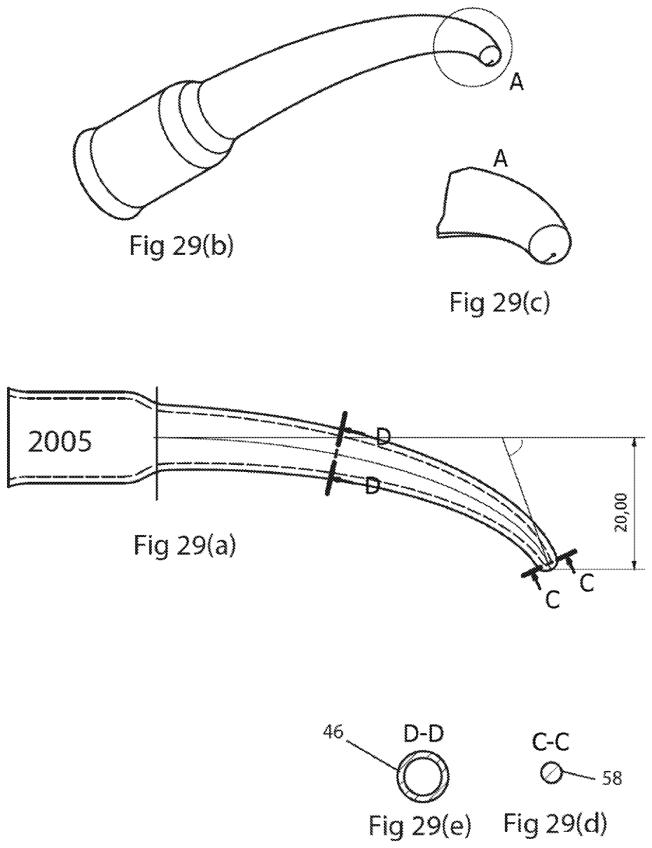

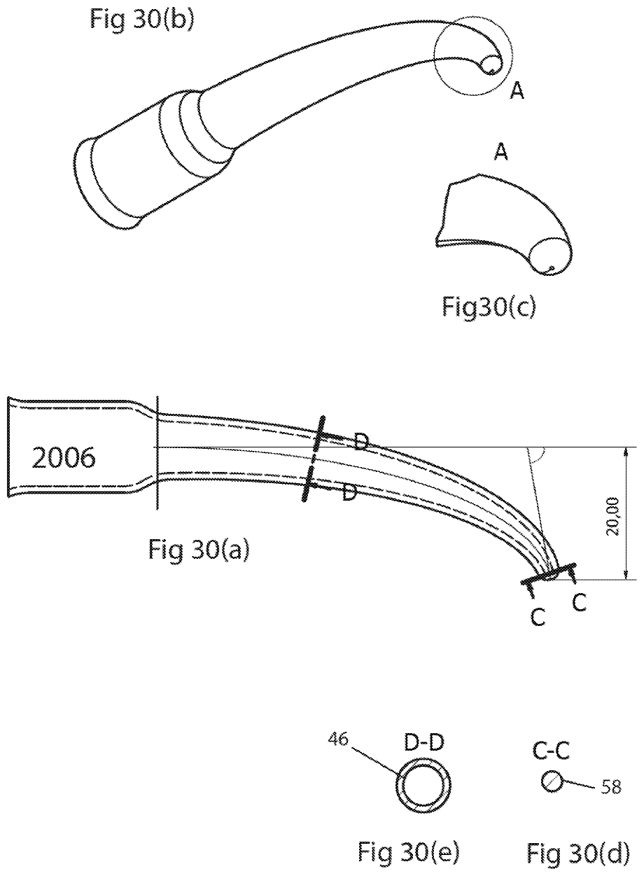

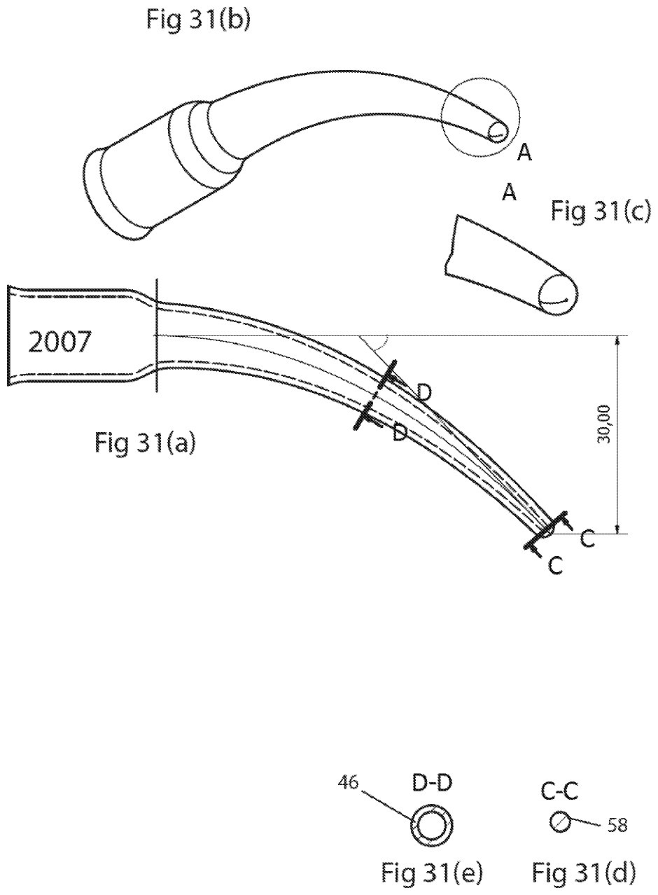

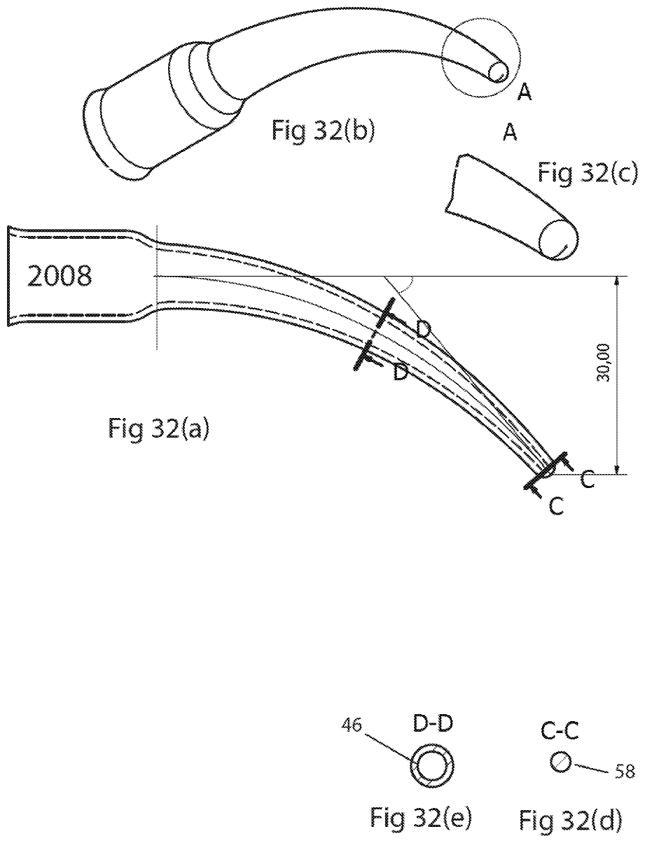

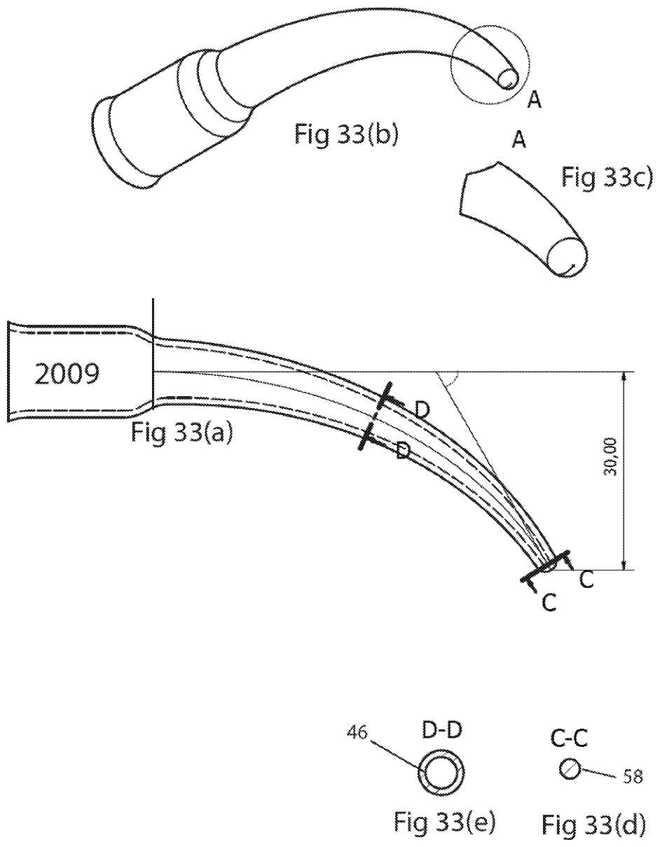

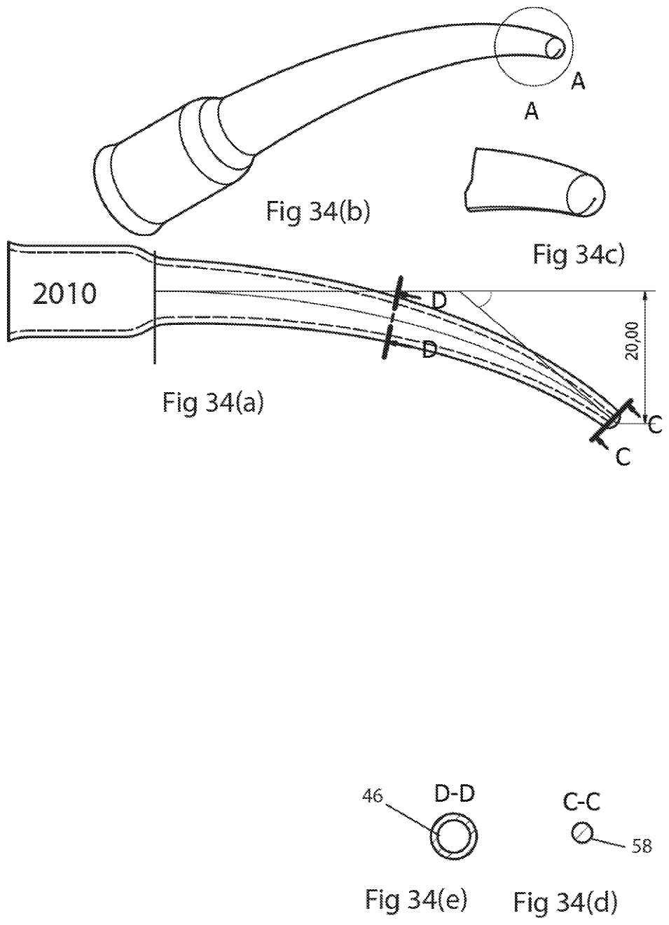

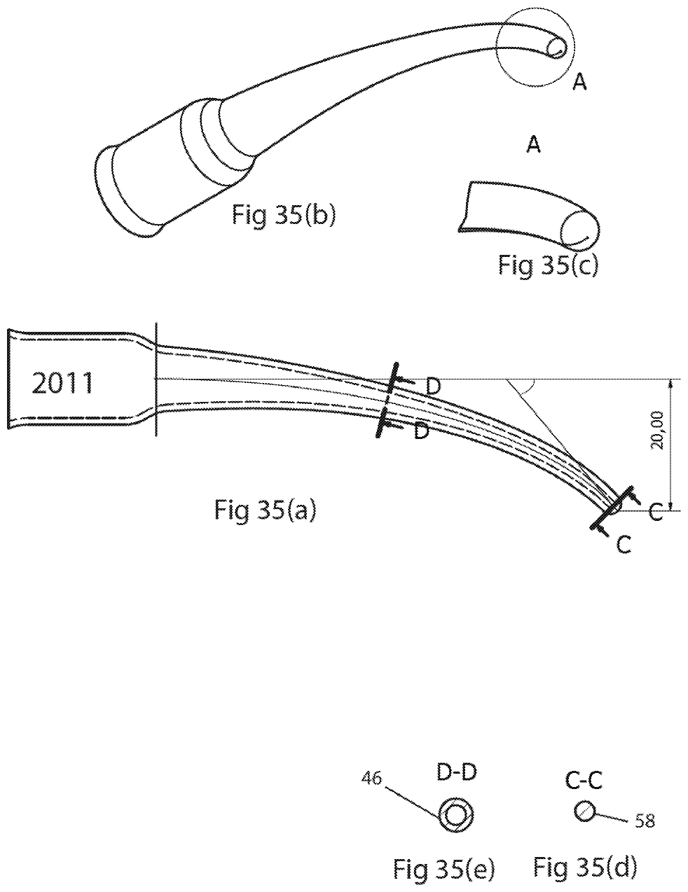

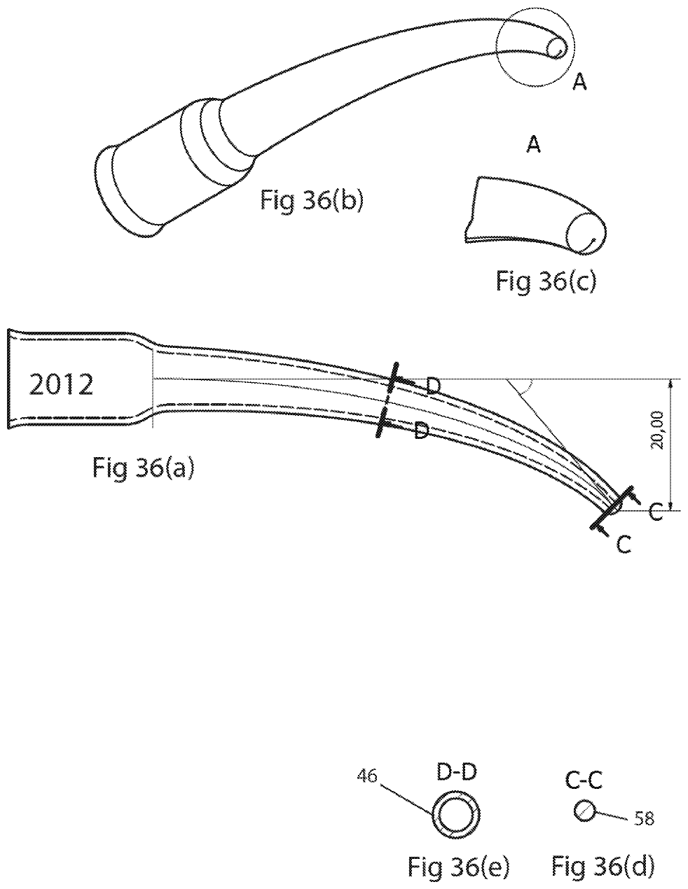

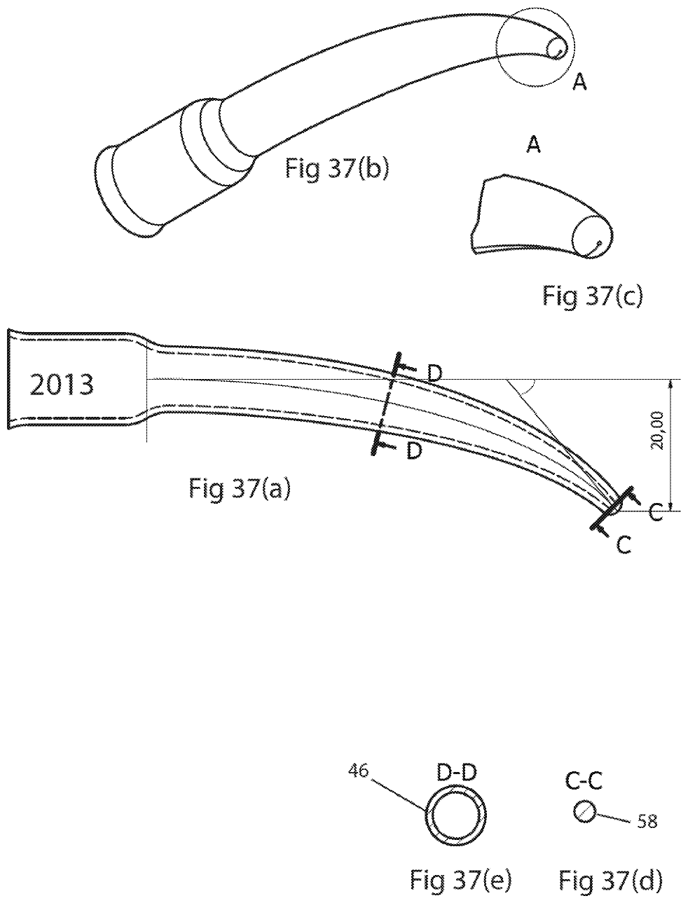

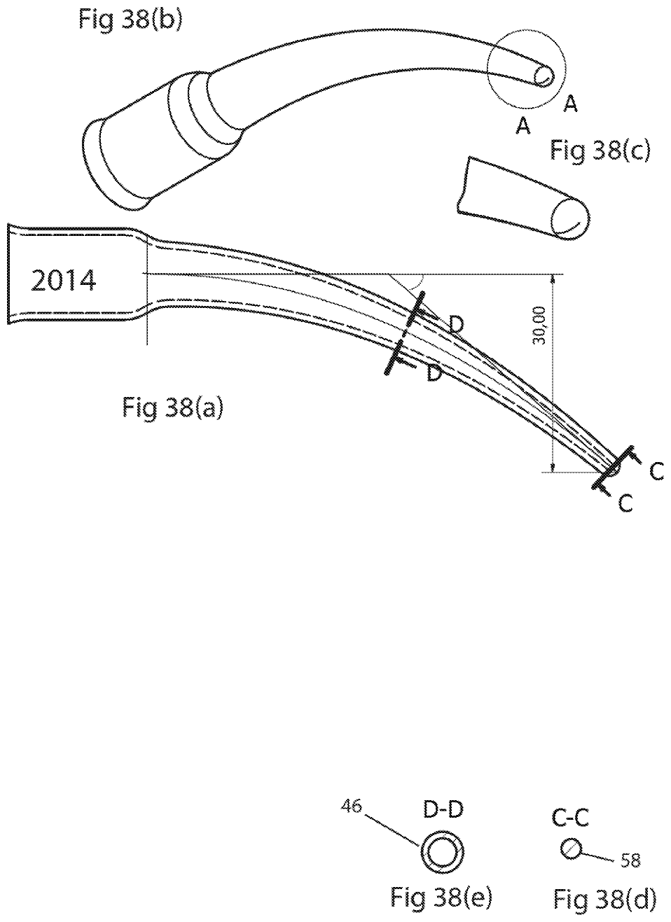

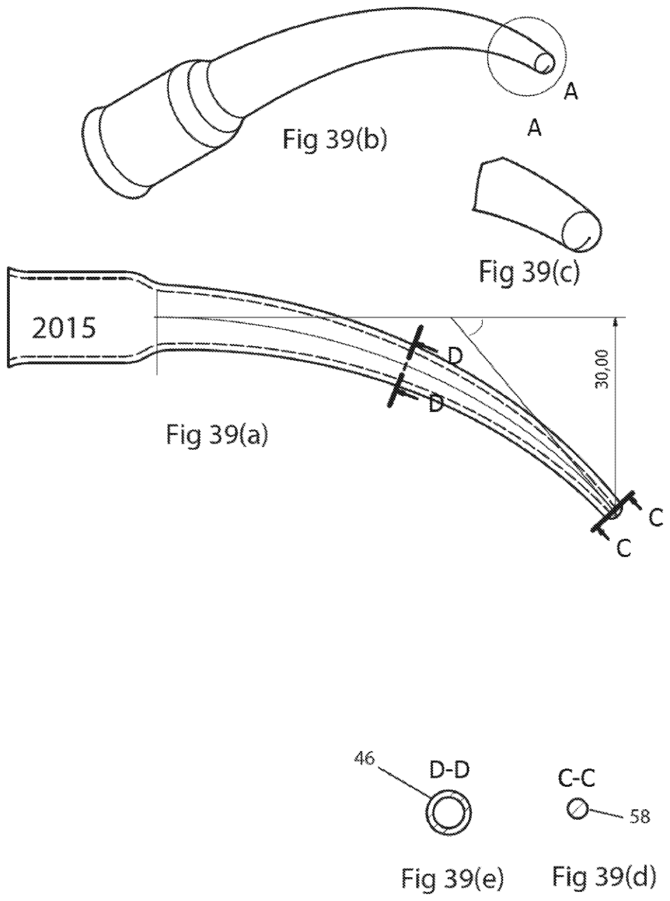

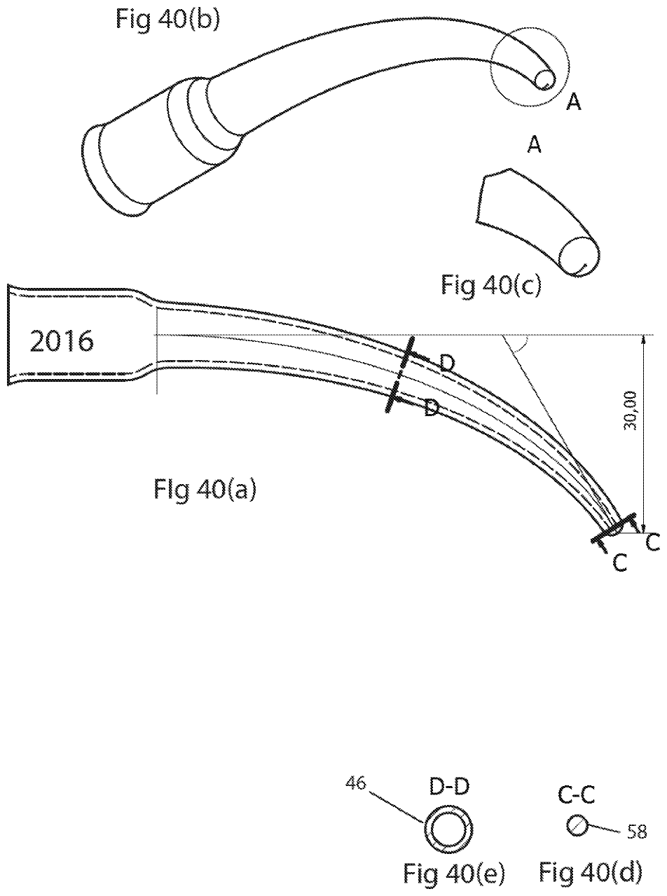

A total of 16 probes were tested, as described in Table 2 and depicted in FIGS. 25 to 40. The printing substrate used was white polylactic acid.

Probe IDs 2001 to 2010 plus 2014 to 2016 were a series to evaluate the effect on ease of use and effectiveness of changing the length, changing the deflection distance, and the tip angle. Prototypes 2011 to 2013 were to evaluate the effect of changing the probe size at the mid point 30.

TABLE 2
Experiment 2 prototypes

| | | | | Mid-Point | | | | |
|---|---|---|---|---|---|---|---|---|
| | Length | Deflection | Tip Angle | (30) | MPP-W | MPP-H | TP-W | TP-H |
| Prototype | (72) | (22) | (32) | % | (48) | (50) | (60) | (62) |
| ID | mm | mm | degrees | L(38) | mm | mm | mm | mm |
| 2001 | 50 | 20 | 50 | 50 | 3.25 | 3.25 | 1.5 | 1.5 |
| 2002 | 50 | 30 | 50 | 50 | 3.25 | 3.25 | 1.5 | 1.5 |
| 2003 | 50 | 30 | 60 | 50 | 3.25 | 3.25 | 1.5 | 1.5 |
| 2004 | 60 | 20 | 50 | 50 | 3.25 | 3.25 | 1.5 | 1.5 |
| 2005 | 60 | 20 | 70 | 50 | 3.25 | 3.25 | 1.5 | 1.5 |
| 2006 | 60 | 20 | 80 | 50 | 3.25 | 3.25 | 1.5 | 1.5 |
| 2007 | 60 | 30 | 40 | 50 | 3.25 | 3.25 | 1.5 | 1.5 |
| 2008 | 60 | 30 | 50 | 50 | 3.25 | 3.25 | 1.5 | 1.5 |
| 2009 | 60 | 30 | 60 | 50 | 3.25 | 3.25 | 1.5 | 1.5 |
| 2010 | 70 | 20 | 40 | 50 | 3.25 | 3.25 | 1.5 | 1.5 |
| 2011 | 70 | 20 | 50 | 50 | 2.5 | 2.5 | 1.5 | 1.5 |
| 2012 | 70 | 20 | 50 | 50 | 3.5 | 3.5 | 1.5 | 1.5 |
| 2013 | 70 | 20 | 50 | 50 | 4.5 | 4.5 | 1.5 | 1.5 |
| 2014 | 70 | 30 | 40 | 50 | 3.25 | 3.25 | 1.5 | 1.5 |
| 2015 | 70 | 30 | 50 | 50 | 3.25 | 3.25 | 1.5 | 1.5 |
| 2016 | 70 | 30 | 60 | 50 | 3.25 | 3.25 | 1.5 | 1.5 |

Each probe was tested three times, in three periods each with a different randomized sequence. A commercially available manikin of a mid-sized adult domestic shorthaired cat (Studio Kite, Sydney, Australia), designed and marketed for teaching veterinary students to place endotracheal tubes, was used for the study.

Each probe was rated for three outcome measures (i) length, (ii) curve, and (iii) ease of intubation with the ET tube.

The results were also as follows;
1. shorter length could be compensated by longer deflection,
2. longer length coupled well with a larger midpoint proportion,
3. white PLA scattered the light well, having a “pearled light globe” effect,
4. a finer tip, for a longer section, would be helpful to improve ease of intubation by decreasing the cross sectional area of the proximal larynx that is occupied by the probe,
5. probes with the tip angle of 40°, and of adequate length, would likely be improved in ease-of-use if the midpoint proportion were larger,
6. probes with midpoint half width 48 and midpoint half height 50 of 4.5 mm were very large and filled the mouth making passing of the endotracheal tube more difficult than the others,
7. of all the probes tested probe 2011, with the smaller midpoint width and midpoint height was the most preferred for ease of intubation.

### Conclusions Drawn:

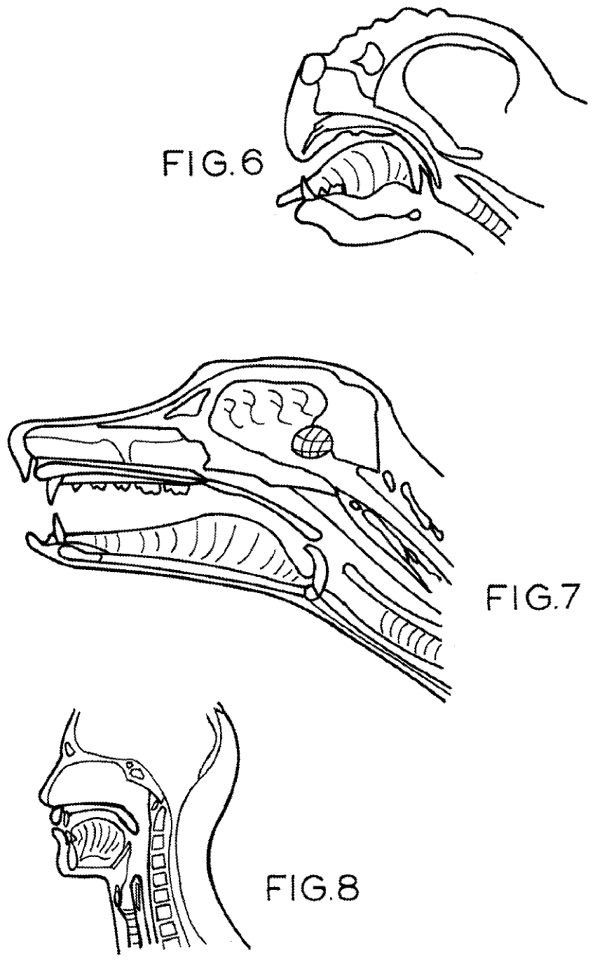

1. the location and shape of the curve is best if it is following the line of the soft palate with the neck in dorsi-flexion. This means that a longer probe for larger cats can be well achieved by lengthening the straight section 26. For other species, the suitable parameter specifications for the probe's shape could be easily developed using a lateral radiographic or computer tomography image or by reference to FIGS. 4 to 8,
2. ensuring that there is space ventral to the probe to pass the endotracheal tube is important. In the current experiment this was clearly enabled by decreasing the midpoint height 50 as was done for probe 2011,
3. constructing the probe so that the whole of it glows like a lightbulb by using translucent materials provided excellent illumination,
4. with the blunt end of the probes used in this series for example 2, it was possible to open the arytenoids, but the tip of the probe was a little large for particularly very easy intubation. A smaller probe tip and altered profiles of midpoint and tip shape may improve ease of intubation,

### Example 3

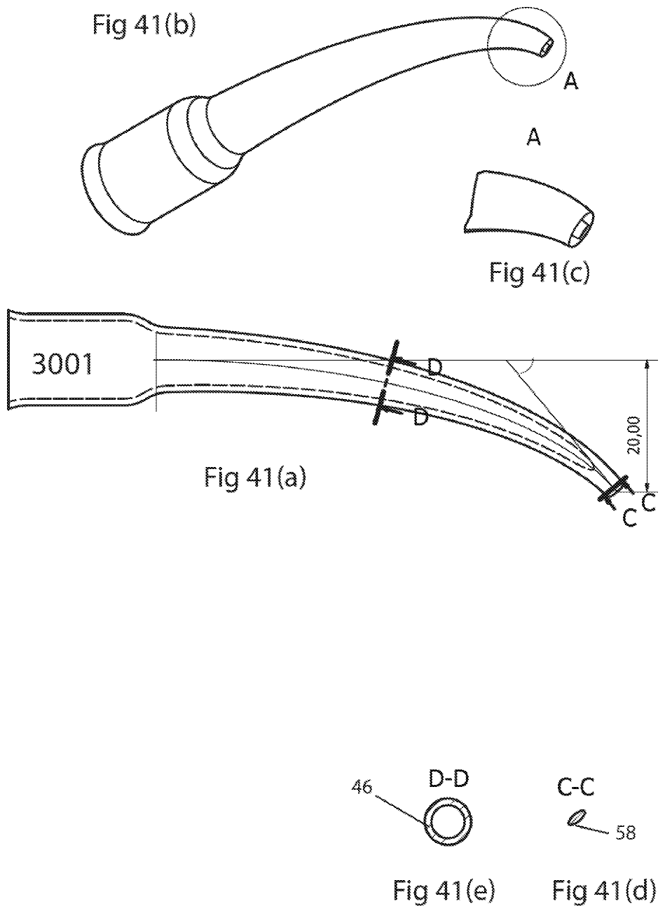

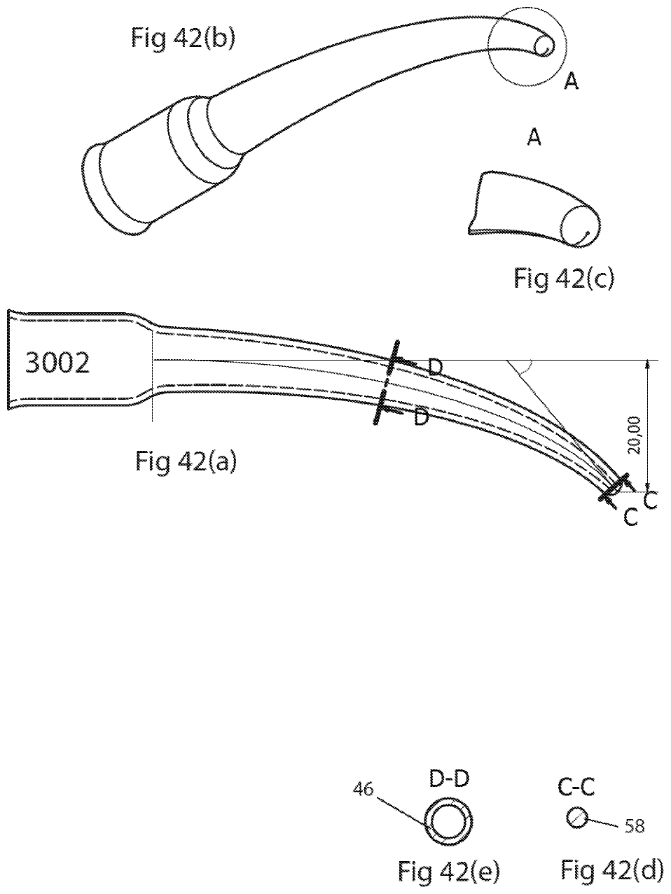

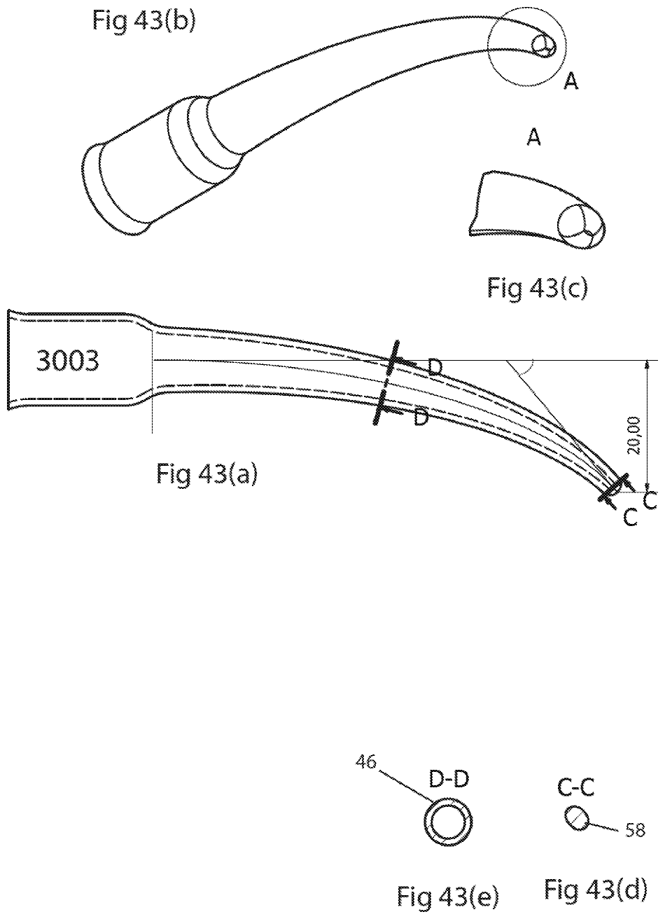

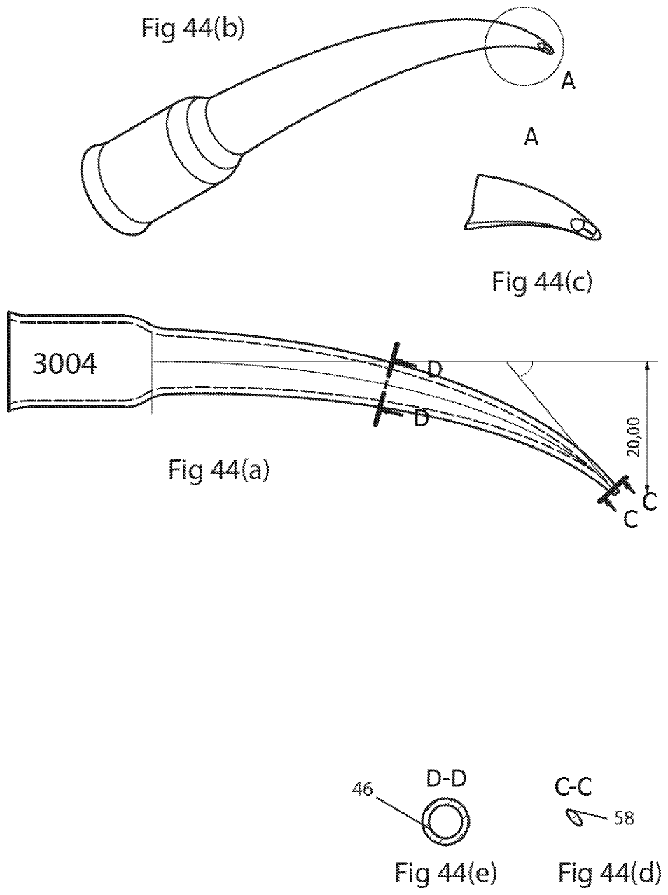

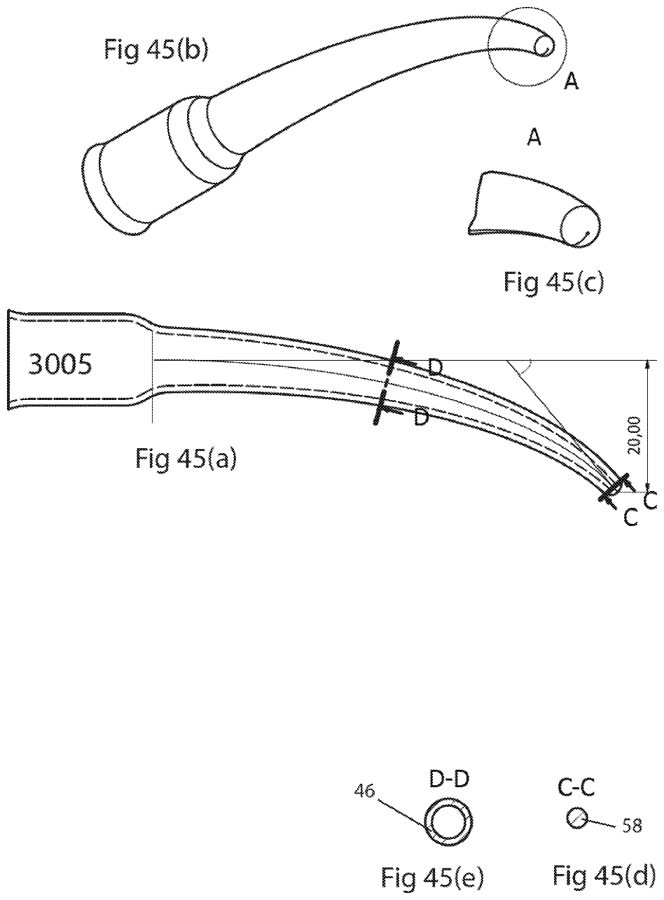

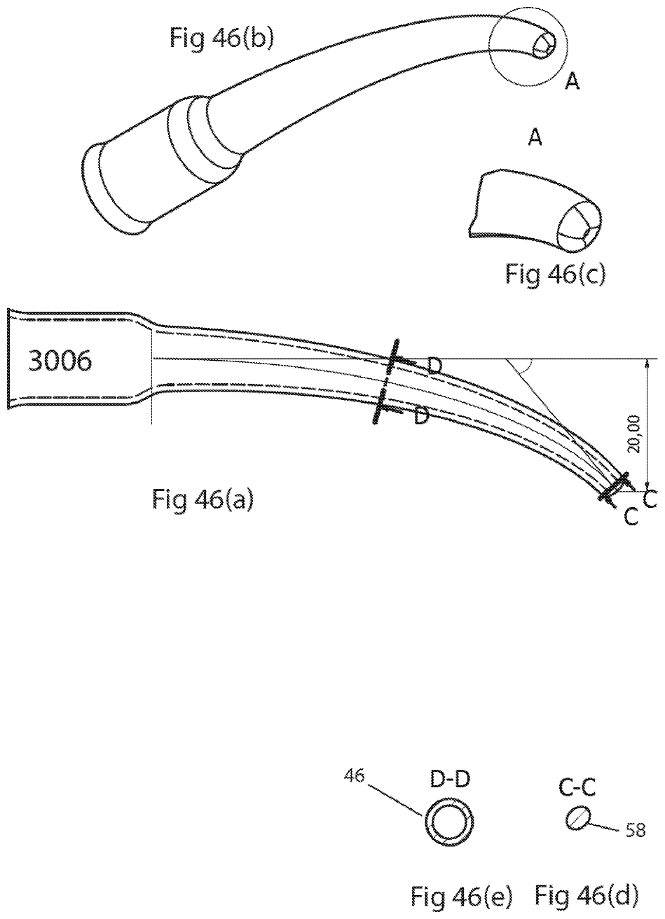

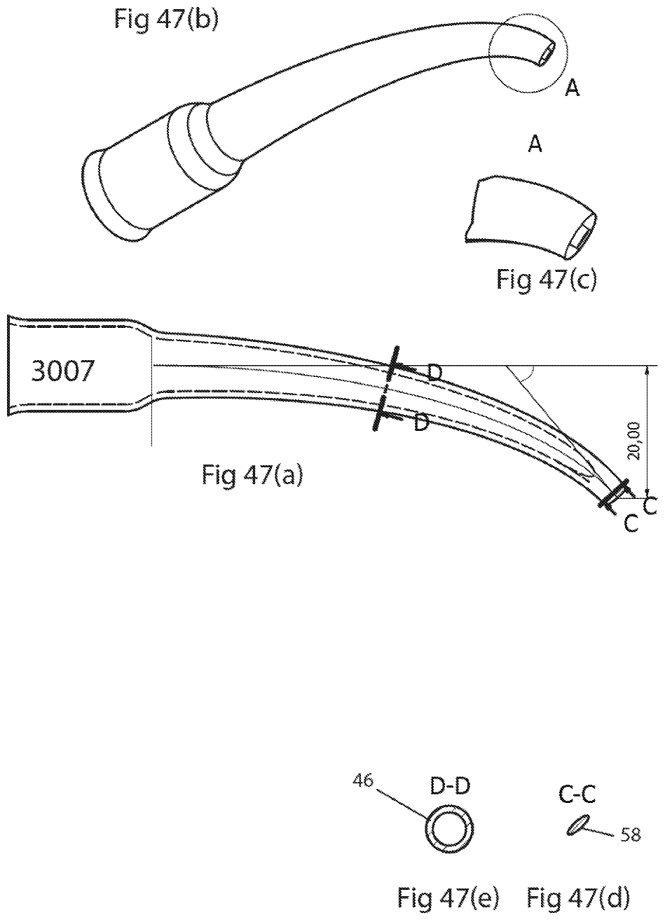

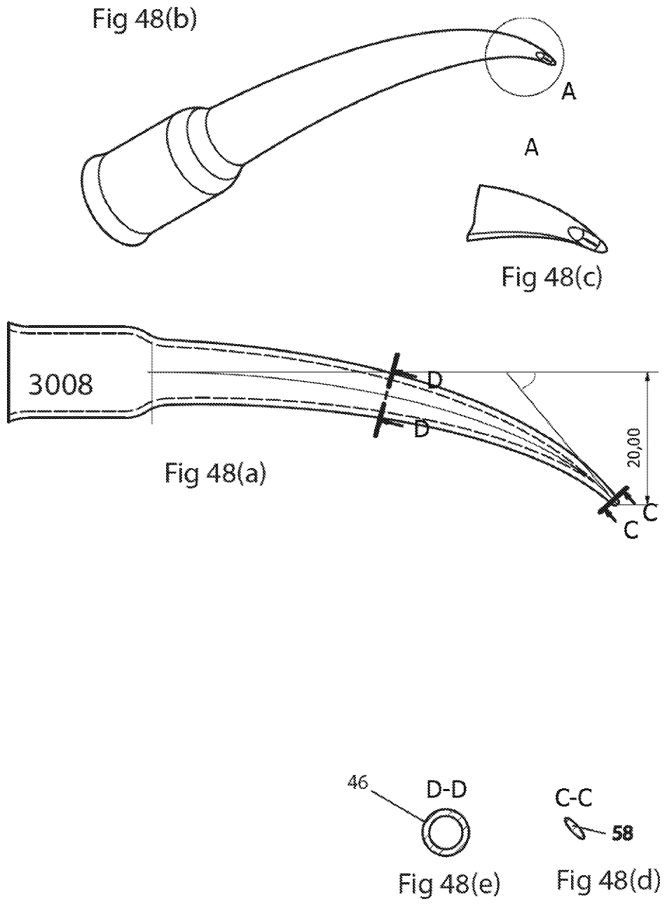

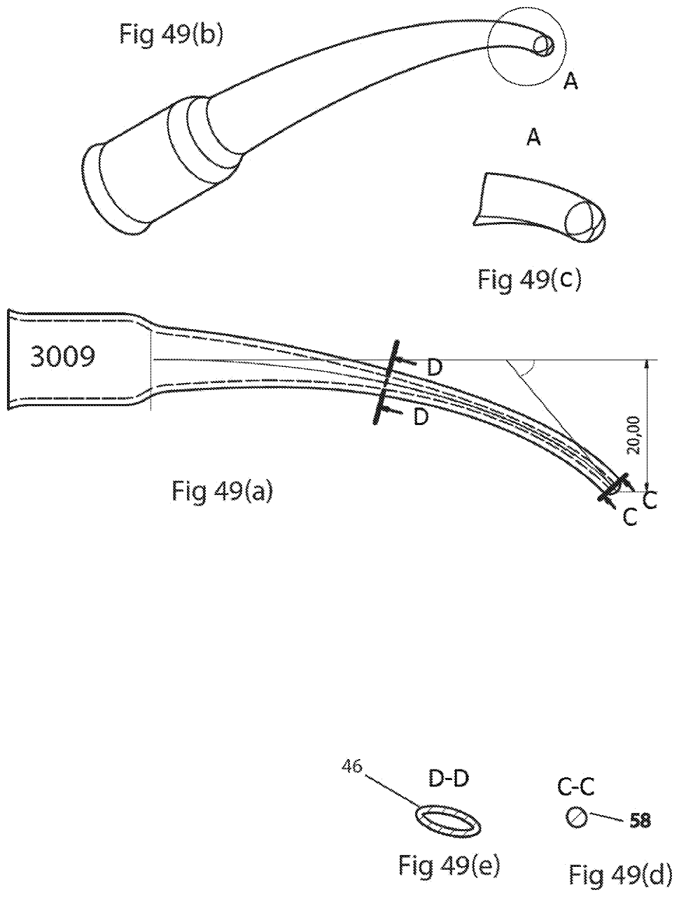

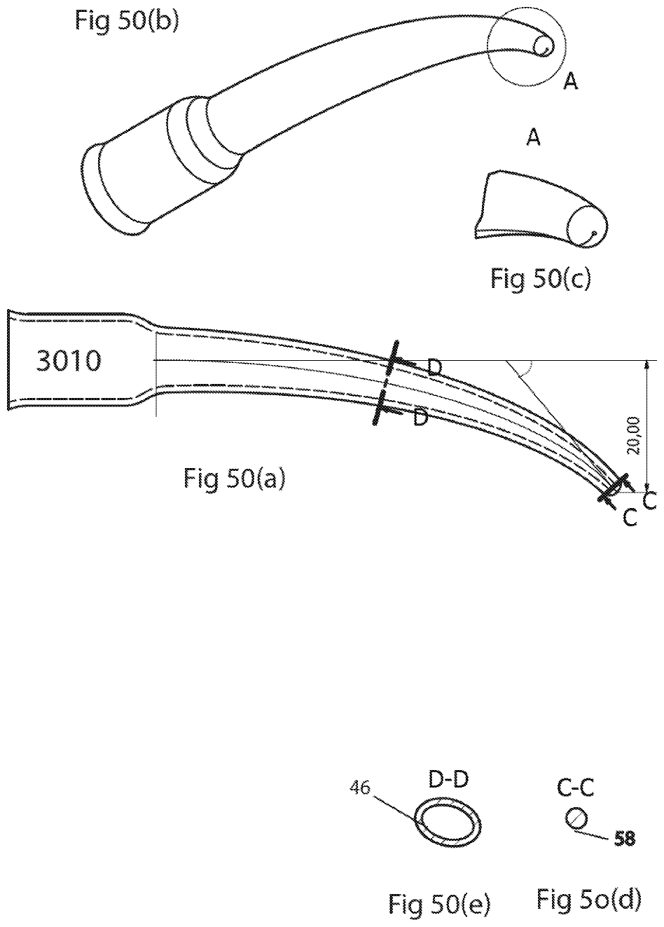

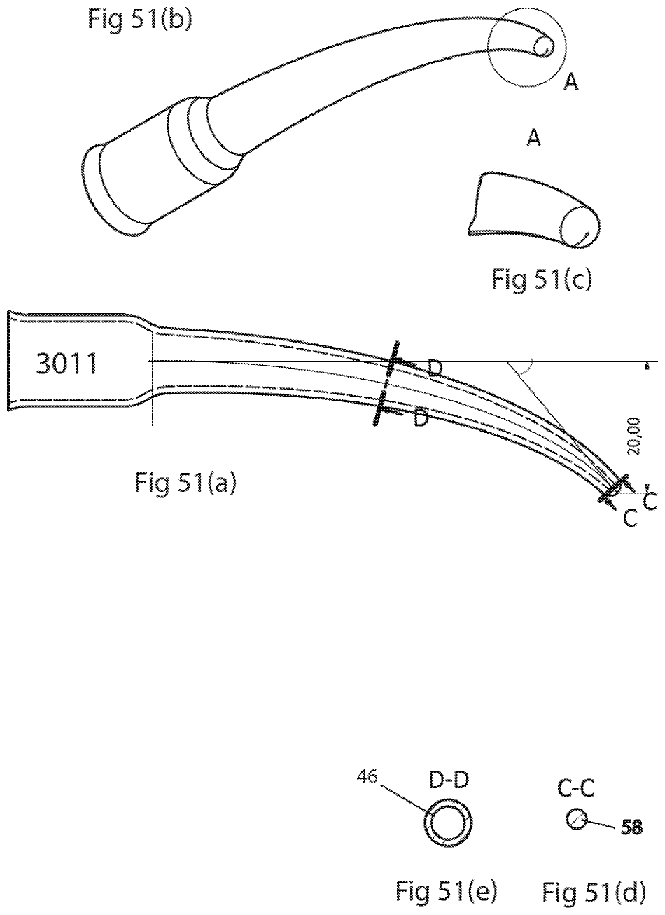

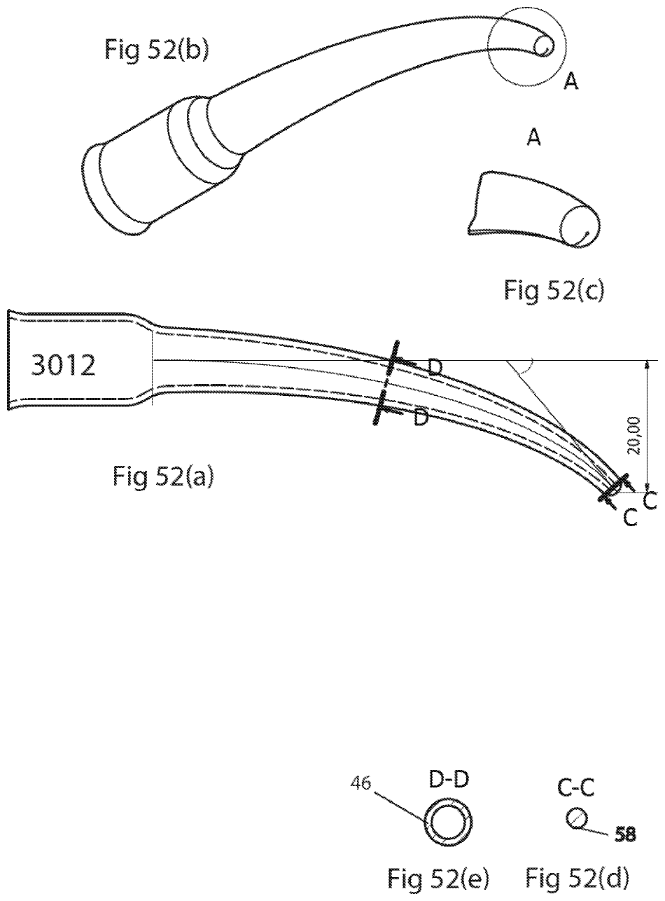

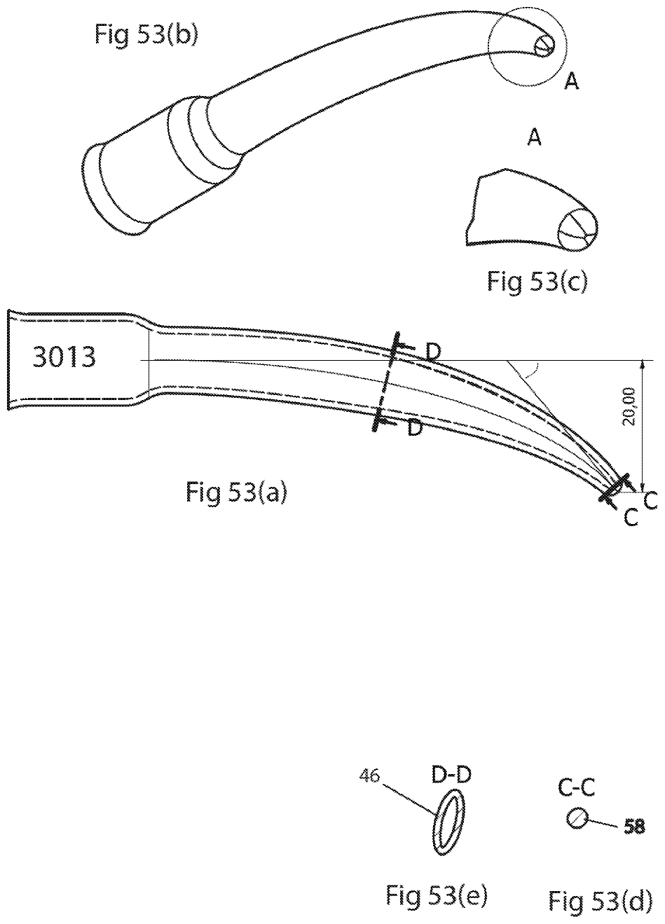

A total of 13 probes tested, as described in Table 3 and depicted in FIGS. 41 to 53. The probes were produced from materials as in Example 2. Each probe was tested once in a randomized sequence. A single period was deemed sufficient because the variability between replicates in Experiment 2 was low. A commercially available manikin of a mid-sized adult domestic shorthaired cat (Studio Kite, Sydney, Australia), designed and marketed for teaching veterinary students to place endotracheal tubes, was used for the study.

TABLE 3
Example 3 blades tested

| | | | | Mid- | | | | |
|---|---|---|---|---|---|---|---|---|
| | | | Tip | Point | MPP- | MPP- | TP- | TP- |
| | Length | Deflection | Angle | (30) | W | H | W | H |
| Prototype | (72) | (34) | (32) | % | (48) | (50) | (60) | (62) |
| ID | mm | mm | Degrees | L(38) | mm | mm | mm | mm |
| 3001 | 70 | 20 | 50 | 50 | 2.5 | 2.5 | 0.5 | 1.5 |
| 3002 | 70 | 20 | 50 | 50 | 2.5 | 2.5 | 1 | 1.5 |
| 3003 | 70 | 20 | 50 | 50 | 2.5 | 2.5 | 1.5 | 1.5 |
| 3004 | 70 | 20 | 50 | 50 | 2.5 | 2.5 | 1.5 | 1 |
| 3005 | 70 | 20 | 50 | 50 | 2.5 | 2.5 | 1.5 | 1.5 |
| 3006 | 70 | 20 | 50 | 50 | 2.5 | 2.5 | 1 | 2 |
| 3007 | 70 | 20 | 50 | 50 | 2.5 | 2.5 | 0.5 | 2 |
| 3008 | 70 | 20 | 50 | 50 | 2.5 | 2.5 | 2 | 0.5 |
| 3009 | 70 | 20 | 50 | 50 | 5 | 2.5 | 1.5 | 1.5 |
| 3010 | 70 | 20 | 50 | 50 | 5 | 2 | 1.5 | 1.5 |
| 3011 | 70 | 20 | 50 | 50 | 5 | 1.5 | 1.5 | 1.5 |
| 3012 | 70 | 20 | 50 | 50 | 4 | 2.5 | 1.5 | 1.5 |
| 3013 | 70 | 20 | 50 | 50 | 4 | 2.5 | 1 | 1 |

The results are as follows;
1. 3001, 3003, 3004, 3005, 3006, 3007 and 3006 each were a more difficult to use than the others because the midpoint profile height 50 partially filled the space ventral to the probe, interfering with passage of the endotracheal tube.
2. 3004 was excellent but would have been improved by decreasing MPP-H 50.
3. 3006, similar to 3001, filled the space ventral to the probe making passage of the endotracheal tube more difficult than some of the others. The gradient of profile change from the tip to the midpoint seemed to be the cause of this problem.
4. The very small and flattened tip of 3008 was excellent for opening the arytenoids and also meant that the size of the probe in the middle was smaller and therefore preferred.
5. 3010 was larger than needed at the midpoint.
6. 3011 was excellent but could have been improved by decreasing the MPP-H 50.
7. The design parameters allowed both the tip 22 and the midpoint 30 to be compressed either laterally or dorsally. While using the probe it was noted that it is possible to rotate the handpiece and thereby change the apparent height to width ratio. This meant that all probes were effective for intubation, but because this required additional fine manipulation, probes with the preferred orientation of width to height without needing user rotation were preferred.
8. Very fine tips are best.
9. The most preferred probe using these design parameters, based on the first three experiments, is likely to be 70-20-50-50-2.5-1.5-1.5-1 (parameters in the order presented in Table 4).

### Conclusions Drawn:

1. A careful user would be able to successfully use any of these probes to open the larynx and passing endotracheal tube, but some sizes and profile shapes a preferred.
2. The tip shape to insert into the fornix of closed arytenoids can be a small and optionally slightly laterally compressed.
3. The profile change near the tip is to flare to dorsally compressed within a few millimeters of the tip. In experiment 3 this was simulated by rotating the probe at that point. There should be no rotation needed, because such rotation changes the fit of the curvature to the soft and hard palates as was establish in experiments 1 and 2.
4. The midpoint of the shaft is best dorsoventrally flattened to make space for the endotracheal tube to be passed ventral to the probe.
5. The design parameterization results in a smooth change in profile from the tip to the midpoint, and from the midpoint to the mount. This means that control of the change in profile close to the tip is lost unless the midpoint percentage is very large. If the midpoint percentage is very large and control of the change of profile along the shaft is lost. Control of the profile change both at the tip and in the middle of the shaft is important.
6. From these first experiments it is concluded that the most preferred probe would comprise a very fine slightly laterally compressed tip which rapidly flares over only 2 or 3 mm of shaft to a dorsoventrally compressed and slightly flared shaft will best allow insertion into the fornix of the arytenoids and then by advancing it quarterly the flare will open the vocal cords. Then by lifting the probe so that it is lying against the dorsal aspect of the larynx the dorsoventral flare will hold the arytenoids completely open. A slight dorsoventrally compressed profile along the shaft of the probe will provide maximum space for visualization and for passage of the endotracheal tube. This shaft profile can change to a circular profile as it approaches the grip 16. This finding identified the need to include additional parameters to define the probe profile changes along its length that are desired, so Near End Point (NE) 28 and NT-W 54 and NT-H 56 and NT % were added to the probe 18 as depicted in FIG. 13.

### Example 4

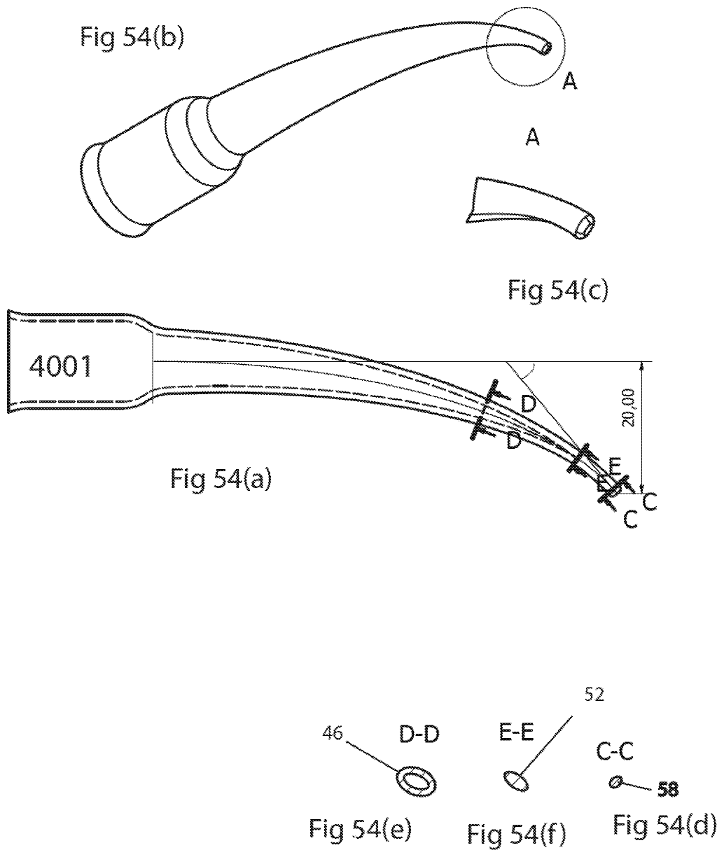

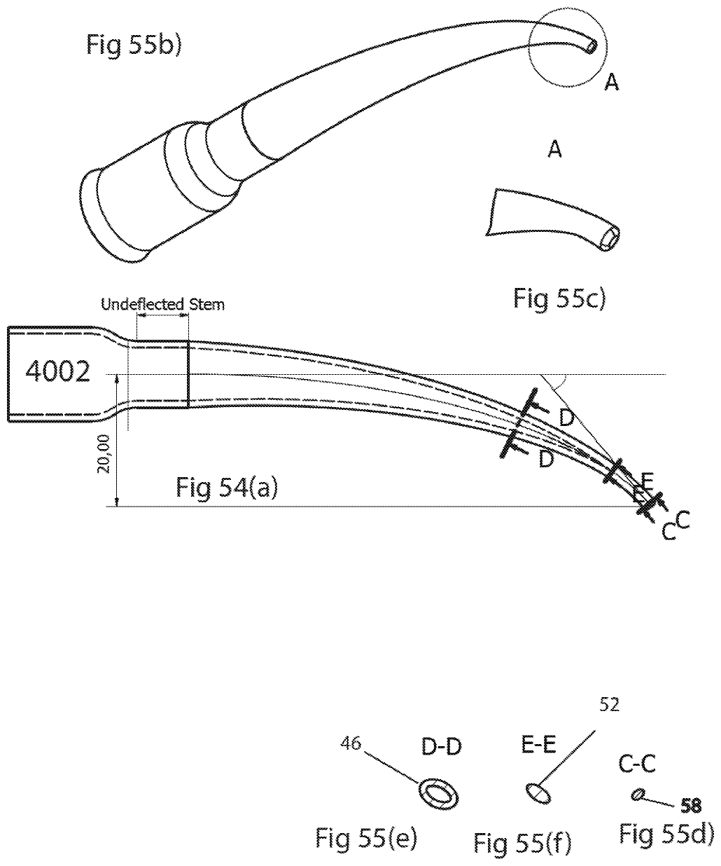

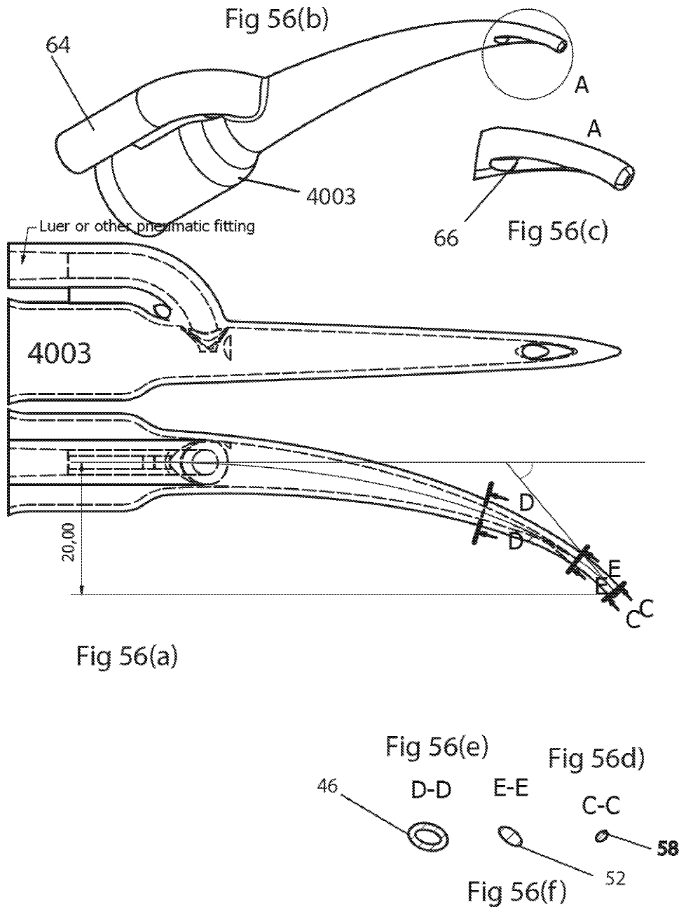

The probes 14 depicted in FIGS. 54 to 56 represent the preferred embodiments of the disclosure with respect to small domestic cats. They each have the following parameters:
Undeflected Length 72—70 mm
Deflection 34—20 mm
Tip Angle 32—50 degrees.

Where they differ is in the location of the mid point, namely, 50% along the deflected length 38 for FIG. 54 (4001) and 70% along the deflected length 38 of FIG. 55 and FIG. 56. FIG. 55 depicts probe no 4003 which is substantially the same as the probe 14 in FIG. 56, namely Probe 4002 except it has a straight portion 26 where the probes 14 in FIGS. 14 to 54 do not have straight portions 26. Further specifications for these preferred embodiments are set out in Table 4 below.

FIG. 56 depicts blade 4002 which is different to all the other described blade 14s.

It is well established in anesthesia of animals that health benefits accrue from ‘pre-oxygenation’ of the animal prior to induction of anesthesia. Pre-oxygenation is the delivery of high partial pressure oxygen instead of air to the animals' breathing, prior to induction of anesthesia. This aims to increase the oxygen saturation of the animal's blood-haemoglobin, allowing for a safer post induction period particularly if apnoea or laryngospasm occurs during the induction of anesthesia.

TABLE 4
Example 4 preferred embodiments tested

| Prototype | Straight | length | Tip | Mid-Point | | | Near-Tip | | | | |
|---|---|---|---|---|---|---|---|---|---|---|---|
| ID | Portion(26) | (72) | Angle | % L | MPP-W | MPP-H | % L | NE-W | NE-H | TP-W | TP-H |
| (units) | mm | mm | degrees | (38) | mm | mm | (38) | mm | mm | mm | mm |
| 4001 | 0 | 70 | 50 | 50 | 3.0 | 2.0 | 90 | 2.0 | 1.0 | 0.5 | 1.0 |
| 4002 | 0 | 70 | 50 | 70 | 3.0 | 2.0 | 90 | 2.0 | 1.0 | 0.5 | 1.0 |
| 4003 | 20 | 70 | 50 | 70 | 3.0 | 2.0 | 90 | 2.0 | 1.0 | 0.5 | 1.0 |

Delivery of high partial pressure oxygen is usually achieved by application of a mask over the animal's nose and mouth, into which oxygen is delivered at high flow rate to displace air and expired gases, for some minutes prior to induction of anesthesia. Upon induction, the mask must be removed in order to gain access to the mouth and larynx for placement of the endotracheal tube. During this time, once again room air is breathed rather than the desired higher oxygen concentration.

TABLE 5
Example and preferred parameter specifications
for probes, optimizing for domestic cats.

| | | lower | most | upper | |
|---|---|---|---|---|---|
| Parameters | minimum | preferred | preferred | preferred | maximum |
| Pre-Curve | 0 | 0 | 0 | 10 | 100 |
| Portion (26) | | | | | |
| Mid-Point (30) | 10 | 40 | 50 | 60 | 90 |
| % Length(38) | | | | | |
| MPP-W (48) | 2 | 2.5 | 3 | 3.5 | 5 |
| MPP-H (50) | 2 | 2 | 2 | 2.5 | 5 |
| Near-End | 70 | 80 | 90 | 95 | 99 |
| % Length(38) | | | | | |
| NE-W (54) | 1 | 1.5 | 2 | 3.5 | 4 |
| NE-H (33) | 0.5 | 0.75 | 1 | 1.5 | 2 |
| TP-W (60) | 0.25 | 0.35 | 0.5 | 0.6 | 1.0 |
| TP-H (62) | 0.8 | 0.9 | 1.0 | 1.5 | 2.0 |
| Undeflected | 40 | 60 | 70 | 120 | 200 |
| Length (72) | | | | | |
| Deflection | 0 | 10 | 20 | 45 | 90 |
| (34) | | | | | |
| Tip Angle (θ) | 0 | 20 | 50 | 70 | 90 |

This modification of the laryngoscope probe 4002 enables it to be used to deliver pure oxygen directly to the larynx, thereby continuing the delivery of higher oxygen content to the animal's breathing. This modification is proposed to use an air-flow channel with a standard medical terminal connection 64 so that it is useful with standard medical tubing and connectors. In this example and medical standard “Luer” fitting is used. It is apparent that any alternative standard sized fitting would be equally suitable. The connector 40 allows attachment to the Probe 14 of a tube carrying, for example, oxygen, which then allows the oxygen to flow through the probe and be delivered from the tip of the probe at tip opening 66 directly into the larynx, thereby increasing the available oxygen for inhalation by the animal, during the process of placing the endotracheal tube. Other gases and vapors could also be delivered through this flow channel for delivery of drugs or gases, such as methoxyflurane or nitrous oxide.

As a result of the experiments conducted the parameters for laryngoscope probes of the present disclosure which has been optimized for domestic cats are set out in Table 5.

The disclosure of the present application has application in a field of commerce including medical sciences and apparatus used by medical specialists to intubate mammals.

1. A laryngoscope for providing a clear and direct view of the larynx during endotracheal intubation of a mammal, comprising:
a pen grip for holding the laryngoscope like a pen,
an elongated probe; attached coaxially with the pen grip, wherein: the elongated probe is elongated and tapered such that a distal tip of the probe has a smallest cross section profile as compared to any cross section of the probe taken between the tip of the probe and a portion of the probe proximate the grip; the elongated probe is curved and deflects away from the axis of probe and pen grip; and the elongated probe is substantially made from a material that transmits light; and
a light source that is directed to the probe, and wherein light incident on the probe is transmitted or scattered in such a way that the probe becomes illuminated and emits light for aid in intubation.

2. The laryngoscope of claim 1, wherein the pen grip incorporates the light source.

3. The laryngoscope of claim 2, wherein the pen grip and the probe are able to be detached, and the pen grip and the probe are attached via frictional forces.

4. (canceled)

5. The laryngoscope of claim 4, wherein the pen grip and light source is a penlight.

6. The laryngoscope of claim 1, wherein the probe is hollow and made from translucent glass, acrylic, plastic or polymeric material.

7. The laryngoscope of claim 6, wherein the probe is adapted to be heat sterilized or autoclaved.

8. The laryngoscope of claim 6, wherein the probe is adapted for single use and disposal.

9. The laryngoscope of claim 5, wherein the probe has a grip portion that extends over the end of the pen light and attaches via frictional forces and a blade portion that extends from the grip portion, wherein the blade portion may include a straight portion extending between the grip portion and a curve start point of the probe.

10. The laryngoscope of claim 9, wherein the tip angle of deflection is between 5 degrees and 90 degrees and wherein the deflection measured in mm is between 5 mm and 90 mm.

11. The laryngoscope of claim 10, wherein the tip angle of deflection is between 20 degrees and 70 degrees and wherein the deflection measured in mm is between 20 mm and 70 mm.

12. (canceled)

13. The laryngoscope of claim 10, wherein the cross-sectional profiles of the curved portion of the blade portion of the probe are elliptical and either dorsally compressed or ventrally compressed or circular.

14. The laryngoscope of claim 13, wherein the cross-sectional profile of the blade portion of the probe at its distal tip is compressed such that it is taller than it is wide to facilitate the probes insertion between the vocal chords or arytenoids of the mammal being intubated.

15-16. (canceled)

17. The laryngoscope of claim 9, wherein the cross-sectional profile of the blade portion flares out at the near tip position of the blade such that it is wider than it is tall which facilitates the opening of the vocal chords of the mammal being intubated.

18-19. (canceled)

20. The laryngoscope of claim 13, wherein at the mid-point of the blade of the probe the cross sectional profile is flattened dorsally with a greater width than height.

21-25. (canceled)

26. The laryngoscope of any of claims 1 to 25 wherein the laryngoscope is hollow and includes: a connection for connecting a medical gas, a passageway through the probe to an aperture located near the distal tip of the probe which communicates the medical gas to the mammal during the use of the laryngoscope, where the medical gas is oxygen and wherein the oxygen is delivered to the pharynx and/or larynx of the mammal.

27. (canceled)

28. A method for intubating a mammal, comprising:
providing a laryngoscope comprising: a pen grip for holding the laryngoscope like a pen; an elongated probe attached coaxially with the pen grip, wherein: the elongated probe is elongated and tapered such that a distal tip of the probe has a smallest cross section profile as compared to any cross section of the probe taken between the tip of the probe and a portion of the probe proximate the grip; the elongated probe is curved and deflects away from the axis of probe and pen grip; and the elongated probe is substantially made from a material that transmits light; and a light source that is directed to the probe, wherein light incident on the probe is transmitted or scattered in such a way that the probe becomes illuminated and emits light for aid in intubation.
with a first hand, holding the grip of the laryngoscope in a pen grip;
passing the probe of the laryngoscope into the buccal cavity of the mammal and along the roof of the mouth in contact with the hard palate;
pushing the probe caudally, dorsal to the epiglottis, lifting the soft palate with the probe;
visualising the proximal larynx;
applying local anesthetic to the larynx;
pushing the lubricated tip of the probe of the laryngoscope into the small fornix of the vocal folds between the arytenoid cartilages;
raising the tip of the probe dorsally whist pushing it caudally thereby opening the arytenoid cartilages;
using a second hand, passing a lubricated endotracheal tube ventral to (underneath) the laryngoscope and into the proximal larynx; and
withdrawing the laryngoscope as the endotracheal tube tip is passed gently between the arytenoids.

29. The method of claim 28 further comprising: inducing general anesthesia; placing the mammal in ventral recumbency; and grasping a tongue of the mammal and pulling it forward presenting the open mouth for intubation.

30. The method of claim 28, further comprising administering oxygen or medical gas to the mammal throughout intubation via an opening in the probe connected to a source of oxygen or medical gas.

31. The method of claim 28, wherein the mammal is a feline.

32. The method of claim 31, wherein the mammal is a domestic cat, a rodent, or a canine.

33. (canceled)
